(19) United States
(12) Patent Application Publication
(10) Pub. No.: US 2026/0364336 A1
(43) Pub. Date: Nov. 19, 2026

(54) REPOSITIONING SHEATH

(71) Applicant: Boston Scientific Scimed, Inc., Maple Grove, MN (US)

(72) Inventors: Daniel Frank Massimini, Brooklyn Park, MN (US); Nicholas Lee Tassoni, Andover, MN (US); Judith Tiferes Wang, Shoreview, MN (US); Neeraj Kumar Sharma, New Delhi (IN)

(73) Assignee: Boston Scientific Scimed, Inc., Maple Grove, MN (US)

(21) Appl. No.: 19/674,651

(22) Filed: May 12, 2026

Related U.S. Application Data
(60) Provisional application No. 63/804,882, filed on May 13, 2025.

(51) Int. Cl. A61M 60/865; A61M 39/02; A61M 60/13; A61M 25/01; A61M 25/06

(52) U.S. Cl. CPC A61M 60/865; A61M 39/0247; A61M 60/13; A61M 25/0113; A61M 2025/0681; A61M 2039/0258; A61M 2039/0273

(57) ABSTRACT

A vascular access sheath is described. The vascular access sheath includes an elongate tubular shaft having a proximal end, a distal end, and a lumen extending therethrough along a longitudinal axis of the elongate shaft. A hub is secured to the proximal end of the elongate tubular shaft. The hub includes an access port in communication with the lumen of the elongate shaft. A support is provided with the hub. The support includes an engagement surface extending at an acute angle to the longitudinal axis of the elongate shaft which is configured to rest directly or indirectly against the skin of a patient to maintain the hub at a fixed, stationary acute angle relative to the skin of the patient.

### CROSS REFERENCE TO RELATED APPLICATIONS

This application claims the benefit of U.S. Provisional Patent Application Serial No. 63/804,882 filed on May 13, 2025, the disclosure of which is incorporated herein by reference.

### TECHNICAL FIELD

The present disclosure pertains to a vascular access sheath configuration for a percutaneous circulatory support system. More particularly, the present disclosure pertains to a vascular access sheath for use at a vascular access site that includes a feature for controlling the angular position of the hub of the vascular access sheath.

### BACKGROUND

In various procedures for delivering intravascular medical devices into a vasculature and/or maintaining a medical device within the vasculature, the intravascular medical device may be advanced through and/or positioned through a vascular access sheath, inserted into a blood vessel of a patient, at an arteriotomy site. In various instances, the medical devices include catheters or other devices, such as a percutaneous blood pump. In some instances, bleeding may occur at the arteriotomy site due to tenting of the artery caused by the angle of the vascular access sheath exiting the arteriotomy site. Accordingly, there is an ongoing need to provide alternative access sheath designs and features to control the angle of the vascular access sheath to mitigate bleeding and/or discomfort at the arteriotomy site.

### BRIEF SUMMARY

This disclosure provides design, material, manufacturing method, and use alternatives for medical devices, including percutaneous blood pumps, vascular access sheaths, and associated devices.

A first example is a vascular access sheath. The vascular access sheath includes an elongate tubular shaft having a proximal end, a distal end, and a lumen extending therethrough along a longitudinal axis of the elongate shaft. A hub is secured to the proximal end of the elongate tubular shaft. The hub includes an access port in communication with the lumen of the elongate shaft. The vascular access sheath also includes a support including an engagement surface configured to rest directly or indirectly against the skin of a patient to maintain the hub at a fixed, stationary acute angle relative to the skin of the patient.

Alternatively or additionally to any of the examples above, the support is rotatable about the longitudinal axis relative to the port of the hub.

Alternatively or additionally to any of the examples above, the support is fixed relative to the port of the hub.

Alternatively or additionally to any of the examples above, the support includes a wedge-shaped body tapering from a wider extent to a narrower extent from the longitudinal axis.

Alternatively or additionally to any of the examples above, the engagement surface lies in a plane extending at an acute angle to the longitudinal axis.

Alternatively or additionally to any of the examples above, the support includes a first wing extending from the wedge-shaped body in a first direction and a second wing extending from the wedge-shaped body in a second, different direction.

Alternatively or additionally to any of the examples above, the engagement surface is a first engagement surface extending at a first acute angle relative to the longitudinal axis, wherein the support includes a second engagement surface extending at a second acute angle relative to the longitudinal axis, the second angle being different from the first acute angle.

Alternatively or additionally to any of the examples above, the support includes a third engagement surface extending at a third acute angle relative to the longitudinal axis, the third angle being different from the first and second acute angles.

Alternatively or additionally to any of the examples above, the support is pyramidal shaped having a triangular base, wherein the first engagement surface extends from a first side of the triangular base, the second engagement surface extends from a second side of the triangular base, and the third engagement surface extends from a third side of the triangular base.

Alternatively or additionally to any of the examples above, the support includes a conically shaped body having a spiral base with an outer periphery that extends eccentrically around the longitudinal axis of the elongate shaft.

Alternatively or additionally to any of the examples above, the engagement surface is a convexly curved engagement surface that varies in distance relative to the longitudinal axis in different radial directions from the longitudinal axis of the elongate shaft.

Alternatively or additionally to any of the examples above, the support is pivotable relative to the port of the hub about a pivot axis extending perpendicular to the longitudinal axis.

Alternatively or additionally to any of the examples above, the support includes first and second arms positioned on opposite sides of the hub.

Alternatively or additionally to any of the examples above, the first and second arms each include an elongate slot, and the hub includes first and second pins extending in opposite directions from the hub, the first pin extending into the elongate slot of the first arm and the second pin extending into the elongate slot of the second arm.

Alternatively or additionally to any of the examples above, the support is slidably disposed within a channel of the hub and movable in a direction generally parallel to the longitudinal axis.

Another example is a vascular access sheath. The vascular access sheath includes an elongate tubular shaft having a proximal end, a distal end, and a lumen extending therethrough along a longitudinal axis of the elongate shaft. A hub is secured to the proximal end of the elongate tubular shaft. The hub includes an access port in communication with the lumen of the elongate shaft. A support is provided with the hub. The support includes an engagement surface extending at an acute angle to the longitudinal axis of the elongate shaft. The engagement surface is configured to rest directly or indirectly against the skin of a patient to maintain the hub at a fixed, stationary acute angle relative to the skin of the patient.

Alternatively or additionally to any of the examples above, the support includes a wedge-shaped body tapering from a wider extent from the longitudinal axis proximate a proximal end of the wedge-shaped body to a narrower extent from the longitudinal axis proximate a distal end of the wedge-shaped body.

Alternatively or additionally to any of the examples above, the support is rotatable about the longitudinal axis relative to the port of the hub.

Yet another example is a method of using a vascular access sheath. The method includes advancing an elongate tubular shaft of the vascular access sheath into a blood vessel of a patient. The vascular access sheath includes a hub secured to a proximal end of the elongate tubular shaft and a support provided with the hub. The method further includes positioning an engagement surface of the support directly or indirectly against the skin of the patient to maintain the hub at a fixed, stationary acute angle relative to the skin of the patient.

Alternatively or additionally to any of the examples above, the support includes a wedge-shaped body.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an illustrative percutaneous circulatory support device including a blood pump;

FIG. 2 is a schematic partial cross-section view of a blood vessel and a heart of a subject with a side view of a portion of an illustrative percutaneous circulatory support device inserted into a blood vessel through a vascular access sheath with a blood pump positioned in the heart;

FIG. 3 is a schematic partial cross-section view of a blood vessel and a side view of an illustrative vascular access sheath having an elongate shaft of a percutaneous circulatory support device extending therethrough;

FIG. 4 is a perspective view of an illustrative vascular access sheath;

FIG. 5 is a side view of the illustrative vascular access sheath of FIG. 4;

FIG. 6 is a perspective view of another illustrative vascular access sheath;

FIG. 7 is a side view of the illustrative vascular access sheath of FIG. 6;

FIG. 8 is a perspective view of another illustrative vascular access sheath;

FIG. 9 is a side view of the illustrative vascular access sheath of FIG. 8;

FIG. 10 is a perspective view of another illustrative vascular access sheath;

FIG. 11 is an end view of the illustrative vascular access sheath of FIG. 10;

FIGS. 12A-12C illustrate a plurality of positions of the vascular access sheath of FIG. 10;

FIG. 13 is a perspective view of another illustrative vascular access sheath;

FIG. 14 is an end view of the illustrative vascular access sheath of FIG. 13;

FIGS. 15A-15C illustrate a plurality of positions of the vascular access sheath of FIG. 13;

FIG. 16 is a perspective view of another illustrative vascular access sheath;

FIGS. 16A-16B illustrate a plurality of positions of the vascular access sheath of FIG. 16;

FIG. 17 is a perspective view of another illustrative vascular access sheath;

FIGS. 17A-17B illustrate a plurality of positions of the vascular access sheath of FIG. 17;

FIGS. 18A-18B illustrate another illustrative vascular access sheath positioned in plurality of positions; and

FIGS. 19A-19B illustrate an illustrative support for positioning a vascular access sheath in a plurality of positions.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

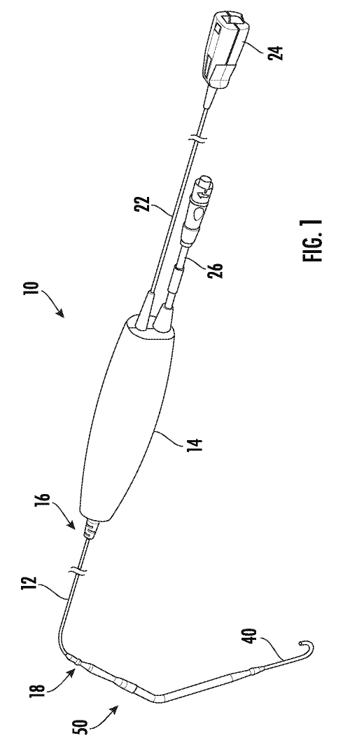

FIG. 1 illustrates a perspective view of a percutaneous circulatory support device 10 including a percutaneous blood pump 50 located at a distal end region thereof. In some instances, the percutaneous circulatory support device 10 may be configured as a percutaneous ventricular assist device (PVAD), a left ventricular assist device (LVAD), or the like. Although other suitable configurations are contemplated, the blood pump 50 may be configured to pump blood from a ventricle of a heart of a subject to vasculature of the subject.

The percutaneous circulatory support device 10 may be coupled to or may include the blood pump 50, with an elongate shaft 12 (e.g., an elongate tube with one or more elongate members extending therein) of the percutaneous circulatory support device 10 coupled with and extending proximally from the percutaneous blood pump 50 and a distal tip 40 extending distally from the blood pump 50. For instance, a proximal end 16 of the elongate shaft 12 may be coupled to a housing 14 (e.g., a housing of a handle, of a control module, etc.) and a distal end 18 of the elongate shaft 12 may be coupled to the percutaneous blood pump 50. An electrical cable 22 may extend from the housing 14 to a connector 24 at a proximal end thereof. The connector 24 may be configured to be connected to a controller (not shown) for controlling the blood pump 50, such as providing electrical power and/or control signals to the blood pump 50. The percutaneous circulatory support device 10 may also include an extension 26 connectable to the controller for sending and/or receiving signals, such as from one or more sensors during operation of the blood pump 50.

When placing the blood pump 50 in a subject (e.g., a patient and/or other suitable subject), the blood pump 50 may be inserted through a vascular access sheath (e.g., an introducer sheath) that extends through a vascular access site in the skin of the subject and into a blood vessel. In some cases, the vascular access sheath may be a large bore sheath configured to receive and facilitate the passage of the blood pump 50 and the elongate shaft 12 through a lumen thereof and into the blood vessel (e.g., a femoral artery, etc.) of the subject. Such an introducer sheath, however, may have an outer diameter that tends to block blood flow through the blood vessel (e.g., flow through the femoral artery to a leg of the subject).

To facilitate perfusion of blood to the leg and/or other portions of the subject, the introducer sheath may be swapped out for or replaced with a repositioning sheath, such as a repositioning sheath having a smaller outer diameter and/or length, after advancing the blood pump 50 into the vasculature of the subject. To facilitate removal of the introducer sheath from the vascular access site (e.g., before, during, or after placement of the repositioning sheath), the introducer sheath may be a peel-away sheath, where the introducer sheath may be configured to split along a perforated line extending along an entirety of or at least part of a length of the introducer sheath, the introducer sheath and/or the repositioning sheath may be configured such that the introducer sheath may slide over the repositioning sheath for removal, and/or the introducer sheath may be configured to be removed from the vascular access site and/or the catheter 10 in one or more other suitable manners. In some examples, the repositioning sheath may have an outer diameter smaller than a diameter of a lumen of the introducer sheath to facilitate placing the repositioning sheath in the blood vessel of the subject through the vascular access site created by the introducer sheath.

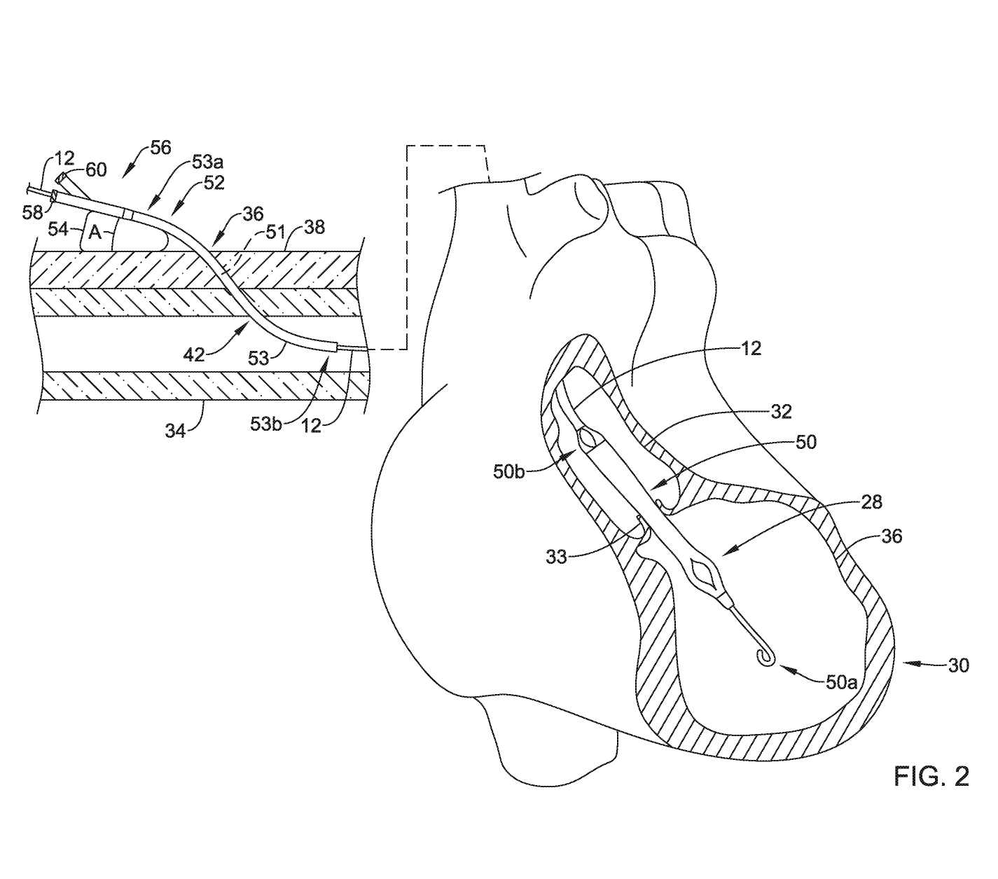

FIG. 2 depicts an illustrative positioning of the blood pump 50 in anatomy of a subject. In FIG. 2, the blood pump 50 is positioned with a distal end 50a located in a left ventricle 28 of a heart 30 and a proximal end 50b in an aorta 32, such that the blood pump 50 extends across an aortic valve 33 between the left ventricle 28 and the aorta 32. With the blood pump 50 extending from the left ventricle 28 to the aorta 32, the blood pump 50 may be configured to pump blood from the left ventricle 28 into the aorta 32 to assist or support blood flow circulation. Other suitable positions of the blood pump 50 relative to the anatomy are contemplated and include, but are not limited to, the distal end 50a of the blood pump 50 being positioned in a right ventricle of the heart 30 with a proximal end 50b being positioned in a pulmonary artery.

As depicted in FIG. 2, the elongate shaft 12 may extend from the blood pump 50 in the aorta 32 through a proximal blood vessel 34 (e.g., the femoral artery and/or other suitable blood vessel) and out of the subject. The elongate shaft 12 may extend through a vascular access sheath 52 (e.g., an introducer sheath, a repositioning sheath, etc.) as the elongate shaft 12 exits the proximal blood vessel 34 and the subject to a location proximal of a hub 56 at a proximal end of an elongate tubular shaft 53 of the vascular access sheath 52, where the elongate tubular shaft 53 may extend through an opening 36 in skin 38 of the subject (e.g., a vasculature access site of the subject) and into the proximal blood vessel 34 through an opening 42 in the proximal blood vessel 34.

The vascular access sheath 52 may have any suitable configuration. In some examples, the vascular access sheath 52 may include the elongate tubular shaft 53 having a proximal end 53a proximate the hub 56, a distal end 53b, and one or more lumens 51 extending between the proximal end and the distal end, along with an outer surface defining an outer diameter of the elongate tubular shaft 53. Additionally or alternatively, at least one of the one or more lumens 51 may extend between one or more openings or ports in the elongate tubular shaft 53 between the proximal end and the distal end.

The hub 56 may have any suitable configuration. In some examples, the hub 56 may include one or more ports. In one example, the hub 56 may include a first port 58 (e.g., a primary port configured to receive the elongate shaft 12 of the percutaneous circulatory support device 10 or other suitable medical device) and a second port 60 (e.g., a second port configured to receive a guidewire or other suitable device). In some examples, the first port 58 and/or the second port 60 may be in communication with one or more lumens 51 of the elongate tubular shaft 53. Other suitable configurations are contemplated.

As the vascular access sheath 52 and the elongate shaft 12 extend proximally out of the subject, it is desirable to retain the hub 56 at an angle to the skin 38 to prevent tenting of the blood vessel 34 and/or bleeding at the opening 36 and/or bleeding under the skin 38 from the opening 42. A support 54 may be positioned between the skin 38 of the subject or other surface supported by the skin 38 of the subject and the hub 56 of the vascular access sheath 52 to maintain a desired angle A between the vascular access sheath 52 and the skin 38 of the subject. For example, the support 54 may be sized or otherwise configured such that the angle A prevents propping open the opening 36 such that the skin 38 may seal around the elongate tubular shaft 53. In some examples, the angle A between the vascular access sheath 52 and the skin 38 of the subject that is maintained by the support 54 therebetween may be at least 30 degrees, at least 40 degrees, or at least 45 degrees, for example. In some examples, the angle A between the vascular access sheath 52 and the skin 38 of the subject that is maintained by the support 54 therebetween may be between about 20 degrees to about 45 degrees, or about 30 degrees to about 45 degrees, for example. Further, the angle A between the vascular access sheath 52 and the skin 38 may be maintained in other suitable manners. Several configurations of the support 54 are described further herein. However, other configurations of the support 54, which may be integrated with the hub 56, formed as a unitary portion with the hub 56, or otherwise supplied with or configured to be used with the hub 56 of the vascular access sheath 52 are contemplated.

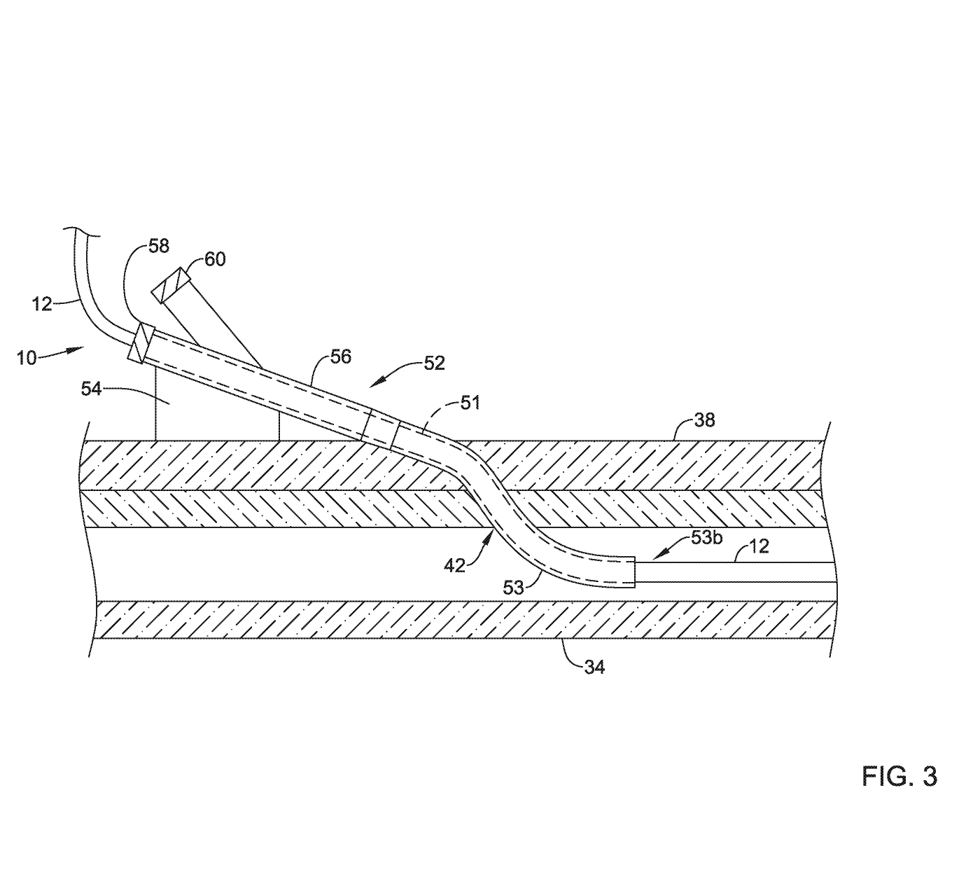

Turning to FIG. 3, as the vascular access sheath 52 may be left in the vasculature of the subject for hours or days (e.g., more than 6 hours, etc.), it can become difficult to maintain the desired angle of the hub 56 relative to the patient’s skin 38 to prevent bleeding at the access site. Accordingly, the various embodiments of a support 54 disclosed herein may provide support of the hub 56 at a fixed, stationary angle relative to the skin 38 of the patient over time as the subject moves and/or when subjected to inadvertent forces. The support 54 may have a surface that is supported against the skin 38 of the patient (e.g., directly contacts the skin 38 of the patient or directly contacts a barrier such as an adhesive patch, a piece of tape or gauze, or the like) to retain the hub 56 at a fixed, stationary angle relative to the skin 38.

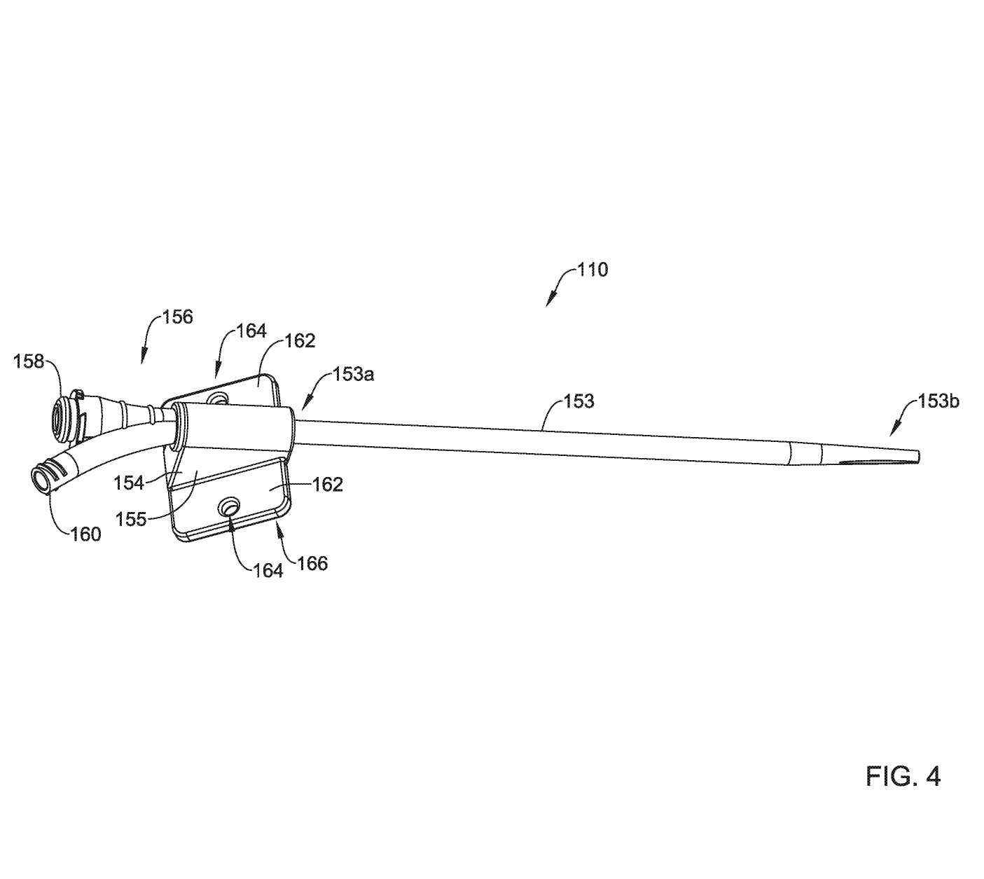

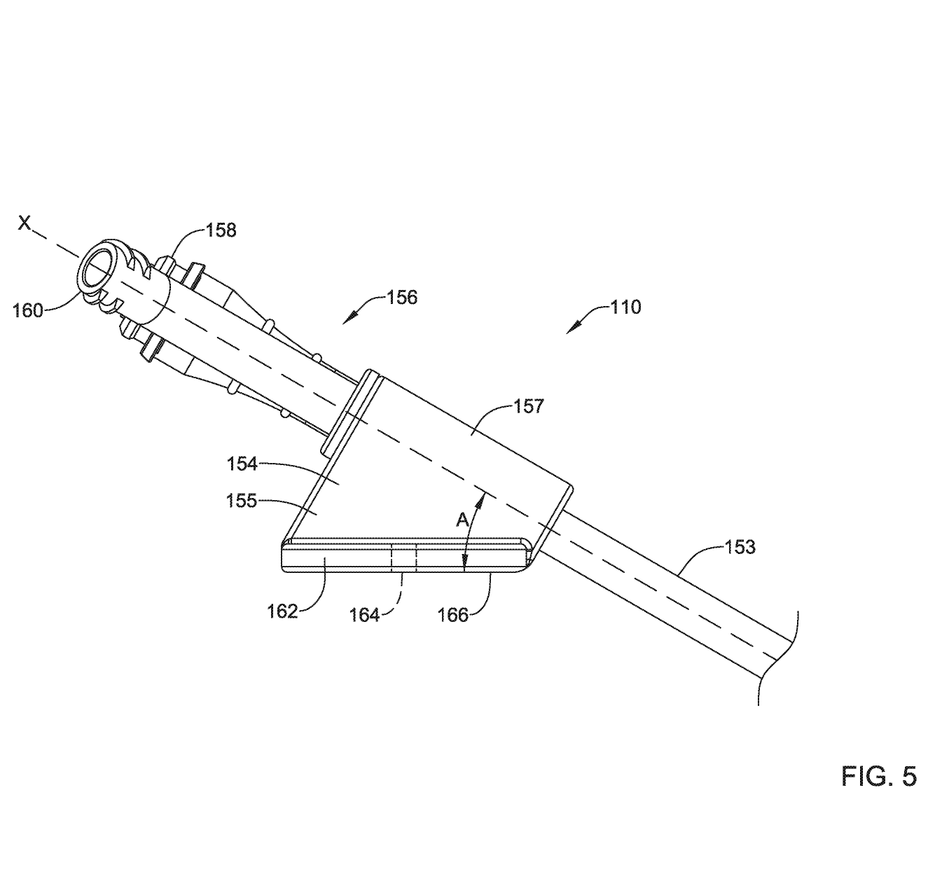

A first embodiment of a vascular access sheath, such as the vascular access sheath 52 described above, incorporating a support for the hub of the vascular access sheath is shown in FIGS. 4 and 5. A vascular access sheath 110 may include a hub 156 and an elongate shaft 153 extending distally from the hub 156 to a distal end 153b of the elongate shaft 153. A proximal end 153a of the elongate shaft 153 may be fixed to the hub 156, or otherwise secured to the hub 156. The hub 156 may define one or more ports providing fluid communication with one or more lumens extending through the elongate shaft 153. For instance, the hub 156 may include a first port 158 in fluid communication with a first lumen of the elongate shaft 153. In some instances, the hub 156 may include a second port 160, which may be in fluid communication with the first lumen of the elongate shaft 153 or in fluid communication with a second lumen of the elongate shaft 153, if desired.

The hub 156 of the vascular access sheath 110 may also include a support 154 configured to rest against (either directly or indirectly) the skin of a patient to maintain the hub 156 at a fixed, stationary acute angle relative to the skin of the patient. In some instances, the support 154 may be fixed relative to the port(s) 158/160 of the hub 156. However, in other instances, the support 154 may be rotatable about the longitudinal axis X of the elongate shaft 153, and thus rotatable relative to the port(s) 158/160. The support 154 may include a wedge-shaped body 155 (e.g., a body tapering from a wider extent to a narrower extent from the longitudinal axis X) and an engagement surface 166 configured to face and/or engage the skin of the patient to retain the hub 156 at a fixed, stationary acute angle relative to the skin of the patient during use of the vascular access device 110. The engagement surface 166 may be configured to extend parallel to and/or lie flush with the skin of the patient with the longitudinal axis X at an acute angle to the skin of the patient. The wedge-shaped body 155 may be any desired structure that orients the engagement surface 166 at an acute angle to the longitudinal axis X of the elongate shaft 153 of the vascular access sheath 110. In some instances, the wedge-shaped body 155 may be a leg extending from a base 157 surrounding the proximal end of the elongate shaft 153. In some instances, the base 157 may be rotatably fixed relative to the elongate shaft 153. However, in other instances, the base 157 may be rotatable relative to the elongate shaft 153, such as rotatable around the longitudinal axis X of the elongate shaft 153.

In the embodiment of FIGS. 4 and 5, the support 154 may include a first wing 162 extending from the wedge-shaped body 155 in a first direction and a second wing 162 extending from the wedge-shaped body 155 in a second, different direction. In some instances, the first direction may be substantially opposite, or opposite the second direction. The engagement surface 166 may be a surface of the first and second wings 162 (e.g., a surface of the pair of wings 162) facing the skin of the patient. The first and second wings 162 may also include openings 164 extending therethrough, which may be configured to allow a suture to be passed therethrough for securing the hub 156 to the skin of a patient.

Referring to FIG. 5, the engagement surface 166 may extend in a plane that intersects the longitudinal axis X of the elongate shaft 153 at a fixed acute angle A. In some examples, the angle A between the longitudinal axis X of the vascular access sheath 110 and the skin of the patient that is maintained by the support 154 positioned therebetween may be between about 20 degrees to about 45 degrees, or about 30 degrees to about 45 degrees, for example.

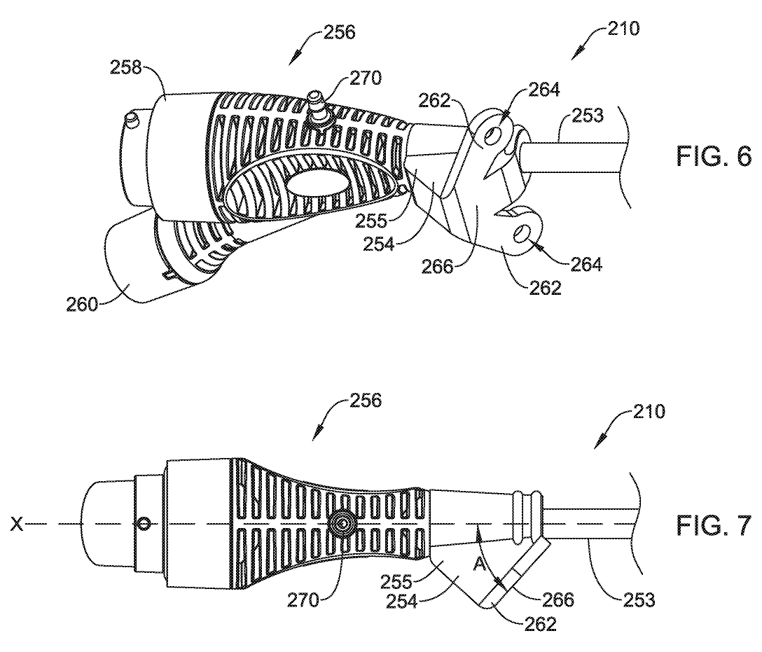

Another embodiment of a vascular access sheath, such as the vascular access sheath 52 described above, incorporating a support for the hub of the vascular access sheath is shown in FIGS. 6 and 7. A vascular access sheath 210 may include a hub 256 and an elongate shaft 253 extending distally from the hub 256 to a distal end of the elongate shaft 253. The proximal end of the elongate shaft 253 may be fixed to the hub 256 or otherwise secured to the hub 256. The hub 256 may define one or more ports providing fluid communication with one or more lumens extending through the elongate shaft 253. For instance, the hub 256 may include a first port 258 in fluid communication with a first lumen of the elongate shaft 253. In some instances, the hub 256 may include a second port 260, which may be in fluid communication with the first lumen of the elongate shaft 253 or in fluid communication with a second lumen of the elongate shaft 253, if desired. The vascular access sheath 210 may also include a flush port 270 in fluid communication with a lumen (e.g., the first lumen or the second lumen) of the elongate shaft 253 to provide the ability to inject a flushing fluid through the lumen, if desired.

The hub 256 of the vascular access sheath 210 may also include a support 254 configured to rest against (either directly or indirectly) the skin of a patient to maintain the hub 256 at a fixed, stationary acute angle relative to the skin of the patient. In some instances, the support 254 may be fixed relative to the port(s) 258/160 of the hub 256. However, in other instances, the support 254 may be rotatable about the longitudinal axis X of the elongate shaft 253, and thus rotatable relative to the port(s) 258/260. The support 254 may include a wedge-shaped body 255 (e.g., a body tapering from a wider extent to a narrower extent from the longitudinal axis X) and an engagement surface 266 configured to face and/or engage the skin of the patient to retain the hub 256 at a fixed, stationary acute angle relative to the skin of the patient during use of the vascular access device 210. The engagement surface 266 may be configured to extend parallel to and/or lie flush with the skin of the patient with the longitudinal axis X at an acute angle to the skin of the patient. The wedge-shaped body 255 may be any desired structure that orients the engagement surface 266 at an acute angle to the longitudinal axis X of the elongate shaft 253 of the vascular access sheath 210. In some instances, the wedge-shaped body 255 may be molded with or integrated with the body of the hub 256 forming the first port 258 and/or the second port 260. In other instances, the wedge-shaped body 253 may be a separate component coupled to the body of the hub 256. In some instances, the wedge-shaped body 255 may be rotatably fixed relative to the elongate shaft 253. However, in other instances, the wedge-shaped body 255 may be rotatable relative to the elongate shaft 253, such as rotatable around the longitudinal axis X of the elongate shaft 253.

In the embodiment of FIGS. 6 and 7, the support 254 may include a first wing 262 extending from the wedge-shaped body 255 in a first direction and a second wing 262 extending from the wedge-shaped body 255 in a second, different direction. The first wing 262 and/or the second wing 262 may extend at any desired angle from the longitudinal axis X, such as at an angle between 0° (i.e., parallel to) to 90° (i.e., perpendicular to) from the longitudinal axis X. In some instances, the first wing 262 may be angled distally toward the distal end of the vascular access sheath 210 at an acute angle to the longitudinal axis X and/or the second wing 262 may be angled distally toward the distal end of the vascular access sheath 210 at an acute angle to the longitudinal axis X. In some instances, the acute angle may be in the range of 10° to 80° form the longitudinal axis X of the elongate shaft 253, in the range of 20° to 60° from the longitudinal axis X of the elongate shaft 253, or in the range of 30° to 45° from the longitudinal axis X of the elongate shaft 253. The first and second wings 262 may also include openings 264 extending therethrough, which may be configured to allow a suture to be passed therethrough for securing the hub 256 to the skin of a patient.

The engagement surface 266 may be a surface of the wedge-shaped body 255 and/or the first and second wings 262 (e.g., a surface of the pair of wings 262) facing the skin of the patient. Referring to FIG. 7, the engagement surface 266 may extend in a plane that intersects the longitudinal axis X of the elongate shaft 253 at a fixed acute angle A. In some examples, the angle A between the longitudinal axis X of the vascular access sheath 210 and the skin of the patient that is maintained by the support 254 positioned therebetween may be between about 20 degrees to about 45 degrees, or about 30 degrees to about 45 degrees, for example. In some instances, the support 254, or at least the first and second wings 262 may be formed of a flexible polymeric material permitting the first and second wings 262 to flex or deform. When suturing the hub 256 to the skin of a patient, the first and second wings 262 may be flexed or deformed toward the skin of the patient, if desired, while the engagement surface 266 defined by the wedge-shaped body 255 therebetween remains in a plane intersecting the longitudinal axis X of the elongate shaft 253 at the acute angle A.

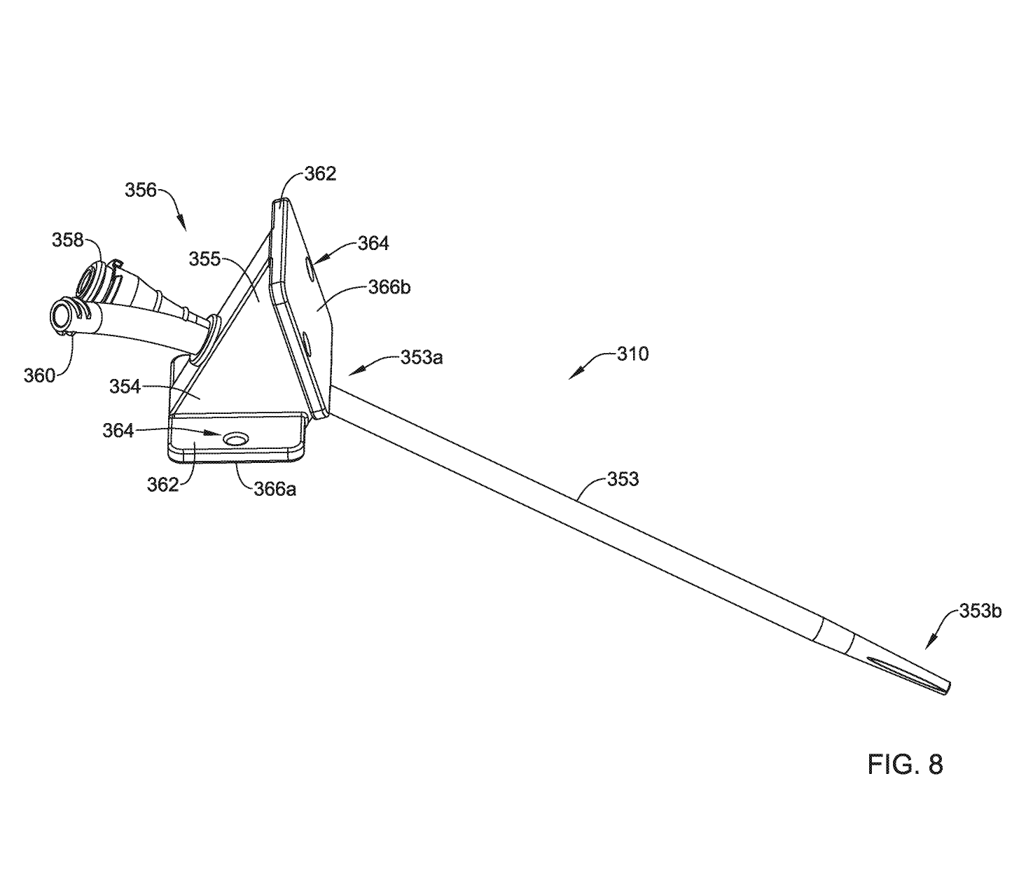

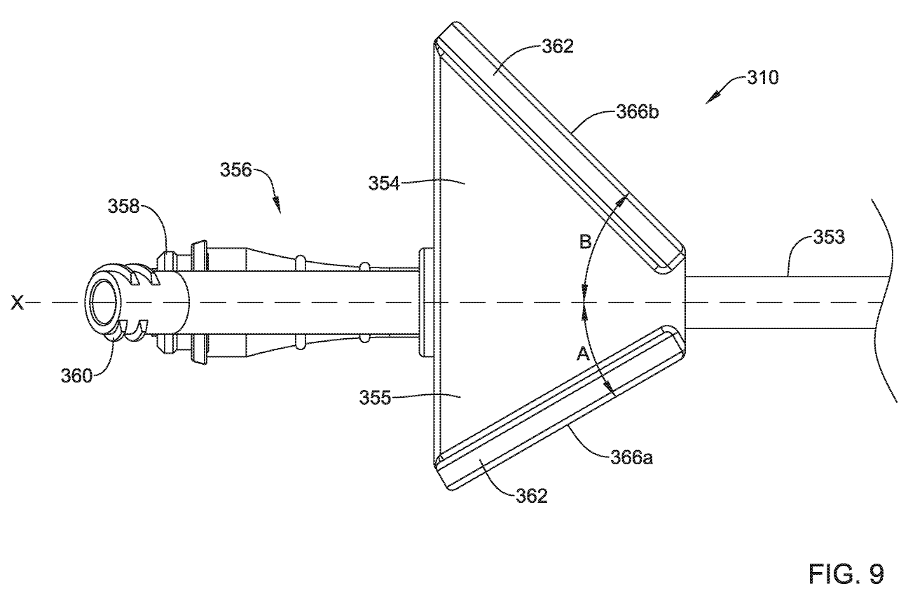

Another embodiment of a vascular access sheath, such as the vascular access sheath 52 described above, incorporating a support for the hub of the vascular access sheath is shown in FIGS. 8 and 9. A vascular access sheath 310 may include a hub 356 and an elongate shaft 353 extending distally from the hub 356 to a distal end 353b of the elongate shaft 353. A proximal end 353a of the elongate shaft 353 may be fixed to the hub 356 or otherwise secured to the hub 356. The hub 356 may define one or more ports providing fluid communication with one or more lumens extending through the elongate shaft 353. For instance, the hub 356 may include a first port 358 in fluid communication with a first lumen of the elongate shaft 353. In some instances, the hub 356 may include a second port 360, which may be in fluid communication with the first lumen of the elongate shaft 353 or in fluid communication with a second lumen of the elongate shaft 353, if desired.

The hub 356 of the vascular access sheath 310 may also include a support 354 configured to rest against (either directly or indirectly) the skin of a patient to maintain the hub 356 at a fixed, stationary acute angle relative to the skin of the patient. In some instances, the support 354 may be fixed relative to the port(s) 358/360 of the hub 356. However, in other instances, the support 354 may be rotatable about the longitudinal axis X of the elongate shaft 353, and thus rotatable relative to the port(s) 358/360. The support 354 may include a wedge-shaped body 355 (e.g., a body tapering from a wider extent to a narrower extent from the longitudinal axis X), a first engagement surface 366a configured to face and/or engage the skin of the patient to retain the hub 356 at a first fixed, stationary acute angle relative to the skin of the patient during use of the vascular access device 310 in a first configuration and/or position, and a second engagement surface 366b configured to face and/or engage the skin of the patient to retain the hub 356 at a second fixed, stationary acute angle relative to the skin of the patient during use of the vascular access device 310 in a second configuration and/or position. The selected engagement surface 366a/366b may be configured to extend parallel to and/or lie flush with the skin of the patient with the longitudinal axis X at an acute angle to the skin of the patient. The wedge-shaped body 355 may be any desired structure that orients the first engagement surface 366a at a first acute angle to the longitudinal axis X of the elongate shaft 353 of the vascular access sheath 310 and that orients the second engagement surface 366b at a second acute angle to the longitudinal axis X of the elongate shaft 353 of the vascular access sheath 310. The first acute angle may be different from the second acute angle, providing the vascular access sheath 310 with a degree of variability by permitting a user to select either the first acute angle or the second acute angle. In some instances, the wedge-shaped body 355 may extend in opposite directions from the longitudinal axis X such that the first engagement surface 366a is located on a first side of a plane extending through and parallel to the longitudinal axis X and the second engagement surface 366b is located on a second side of the plane extending through and parallel to the longitudinal axis X. In some instances, the support 354 (e.g., the wedge-shaped body 355) may be rotatably fixed relative to the elongate shaft 353. However, in other instances, the support 354 (e.g., the wedge-shaped body 355) may be rotatable relative to the elongate shaft 353, such as rotatable around the longitudinal axis X of the elongate shaft 353.

In the embodiment of FIGS. 8 and 9, the support 354 may include first and second wings 362 extending in opposite directions from the wedge-shaped body 355 that define or include the first engagement surface 366a. The first engagement surface 366a may be a surface of the first and second wings 362 (e.g., a surface of the pair of wings 362) facing the skin of the patient when the support 354 is in a first configuration and/or position during use. The support 354 may also include first and second wings 362 extending in opposite directions from the wedge-shaped body 355 that define or include the second engagement surface 366b. The second engagement surface 366b may be a surface of the first and second wings 362 (e.g., a surface of the pair of wings 362) facing the skin of the patient when the support 354 is in a second configuration and/or position during use. One or more, or each of the wings 362 may also include openings 364 extending therethrough, which may be configured to allow a suture to be passed therethrough for securing the hub 356 to the skin of a patient.

Referring to FIG. 9, the first engagement surface 366a may extend in a plane that intersects the longitudinal axis X of the elongate shaft 353 at a first fixed acute angle A. In some examples, the angle A between the longitudinal axis X of the vascular access sheath 310 and the skin of the patient that is maintained by the support 354 positioned therebetween may be between about 20 degrees to about 45 degrees, about 20 degrees to about 40 degrees, or about 30 degrees to about 40 degrees, for example. In some instances, the first angle A may be 30 degrees, 35 degrees or 40 degrees, for example. Furthermore, the second engagement surface 366b may extend in a plane that intersects the longitudinal axis X of the elongate shaft 353 at a second fixed acute angle B, different from the first angle A. In some examples, the angle B between the longitudinal axis X of the vascular access sheath 310 and the skin of the patient that is maintained by the support 354 positioned therebetween may be between about 20 degrees to about 45 degrees, about 30 degrees to about 45 degrees, or about 35 degrees to about 45 degrees, for example. In some instances, the second angle B may be 35 degrees, 40 degrees, or 45 degrees, for example.

During use of the vascular access device 310, the user may determine which of the first and second engagement surfaces 366a/366b to orient towards the skin of the patient to support the hub 356 relative to the skin of the patient in order to provide a desired angle of the hub 356 relative to the skin of the patient. In instances in which the support 354 (e.g., the wedge-shaped body 355) is rotatably fixed relative to the elongate shaft 353, the entire vascular access device 310 may be rotated about the longitudinal axis X in order to orient either the first engagement surface 366a or the second engagement surface 366b toward the skin of the patient. In instances in which the support 354 (e.g., the wedge-shaped body 355) is rotatable relative to the elongate shaft 353, such as rotatable around the longitudinal axis X of the elongate shaft 353, the support 354 may be rotated to orient either the first engagement surface 366a or the second engagement surface 366b toward the skin of the patient. Thus, the support 354 may be used to selectively support the hub 356 of the vascular access sheath 310 at any one of a plurality of fixed, stationary angles relative to the skin of the patient.

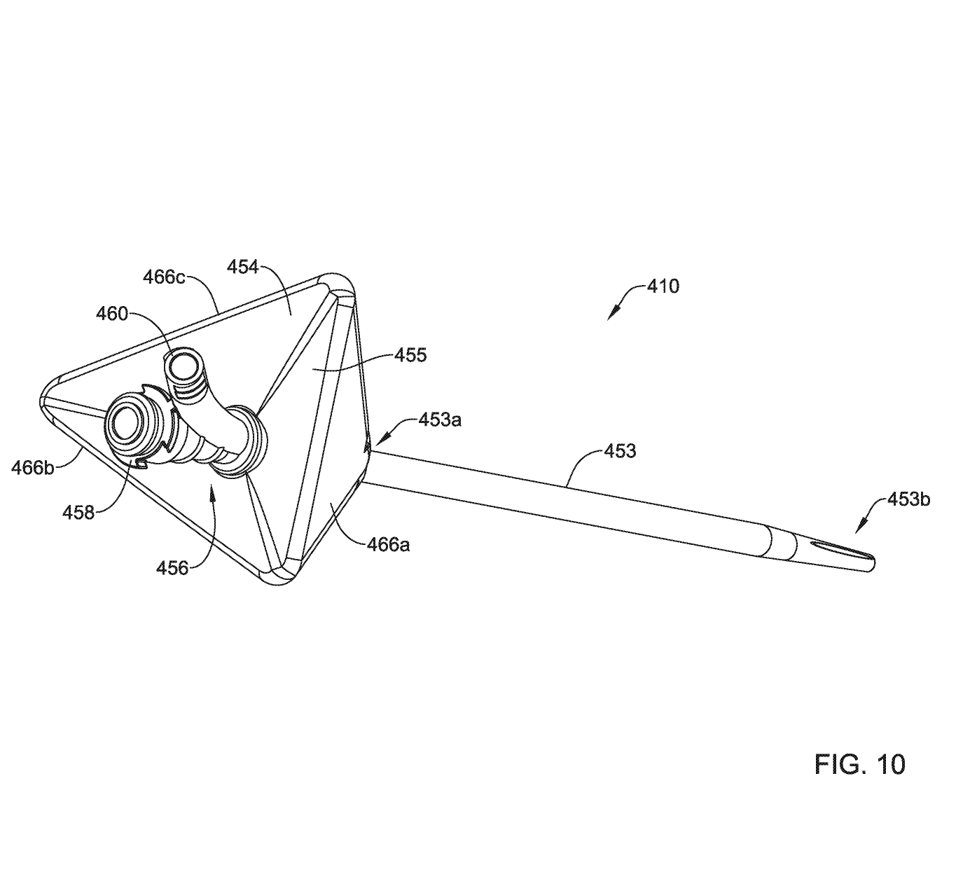

Another embodiment of a vascular access sheath, such as the vascular access sheath 52 described above, incorporating a support for the hub of the vascular access sheath is shown in FIGS. 10, 11 and 12A-12C. A vascular access sheath 410 may include a hub 456 and an elongate shaft 453 extending distally from the hub 456 to a distal end 453b of the elongate shaft 453. A proximal end 453a of the elongate shaft 453 may be fixed to the hub 456 or otherwise secured to the hub 456. The hub 456 may define one or more ports providing fluid communication with one or more lumens extending through the elongate shaft 453. For instance, the hub 456 may include a first port 458 in fluid communication with a first lumen of the elongate shaft 453. In some instances, the hub 456 may include a second port 460, which may be in fluid communication with the first lumen of the elongate shaft 453 or in fluid communication with a second lumen of the elongate shaft 453, if desired.

The hub 456 of the vascular access sheath 410 may also include a support 454 configured to rest against (either directly or indirectly) the skin of a patient to maintain the hub 456 at a fixed, stationary acute angle relative to the skin of the patient. In some instances, the support 454 may be fixed relative to the port(s) 458/460 of the hub 456. However, in other instances, the support 454 may be rotatable about the longitudinal axis X of the elongate shaft 453, and thus rotatable relative to the port(s) 458/460. The support 454 may include a wedge-shaped body 455 (e.g., a body tapering from a wider extent to a narrower extent from the longitudinal axis X), which in some instances may be a pyramid shaped body having a triangular base. The wedge-shaped body 455 may include a plurality of engagement surfaces, each configured to face and/or engage the skin of the patient to retain the hub 456 at one of a plurality of different acute angles relative to the skin of the patient. For example, the wedge-shaped body 455 may include or define a first engagement surface 466a configured to face and/or engage the skin of the patient to retain the hub 456 at a first fixed, stationary acute angle relative to the skin of the patient during use of the vascular access device 410 in a first configuration and/or position, a second engagement surface 466b configured to face and/or engage the skin of the patient to retain the hub 456 at a second fixed, stationary acute angle relative to the skin of the patient during use of the vascular access device 410 in a second configuration and/or position, and a third engagement surface 466c configured to face and/or engage the skin of the patient to retain the hub 456 at a third fixed, stationary acute angle relative to the skin of the patient during use of the vascular access device 410 in a third configuration and/or position. The selected engagement surface 466a/466b/466c may be configured to extend parallel to and/or lie flush with the skin of the patient with the longitudinal axis X at an acute angle to the skin of the patient. The wedge-shaped body 455 may be any desired structure that selectively orients the first engagement surface 466a at a first acute angle to the longitudinal axis X of the elongate shaft 453 of the vascular access sheath 410, orients the second engagement surface 466b at a second acute angle to the longitudinal axis X of the elongate shaft 453 of the vascular access sheath 410, or orients the third engagement surface 466c at a third acute angle to the longitudinal axis X of the elongate shaft 453 of the vascular access sheath 410. Each of the first, second and third acute angles may be different from the others. The first acute angle may be different from each of the second acute angle and the third acute angle, the second acute angle may be different from each of the first acute angle and the third acute angle, and the third acute angle may be different from each of the first acute angle and the second acute angle, providing the vascular access sheath 410 with a degree of variability by permitting a user to select any one of the first acute angle, the second acute angle, or the third acute angle. In some instances, the wedge-shaped body 455 has a triangular base with the first engagement surface 466a extending from a first side of the triangular base along a first plane that intersects the longitudinal axis X at a first acute angle, the second engagement surface 466b extending from a second side of the triangular base along a second plane that intersects the longitudinal axis X at a second acute angle, and the third engagement surface 466c extending from a third side of the triangular base along a third plane that intersects the longitudinal axis X at a third acute angle. In some instances, the support 454 (e.g., the wedge-shaped body 455) may be rotatably fixed relative to the elongate shaft 453. However, in other instances, the support 454 (e.g., the wedge-shaped body 455) may be rotatable relative to the elongate shaft 453, such as rotatable around the longitudinal axis X of the elongate shaft 453.

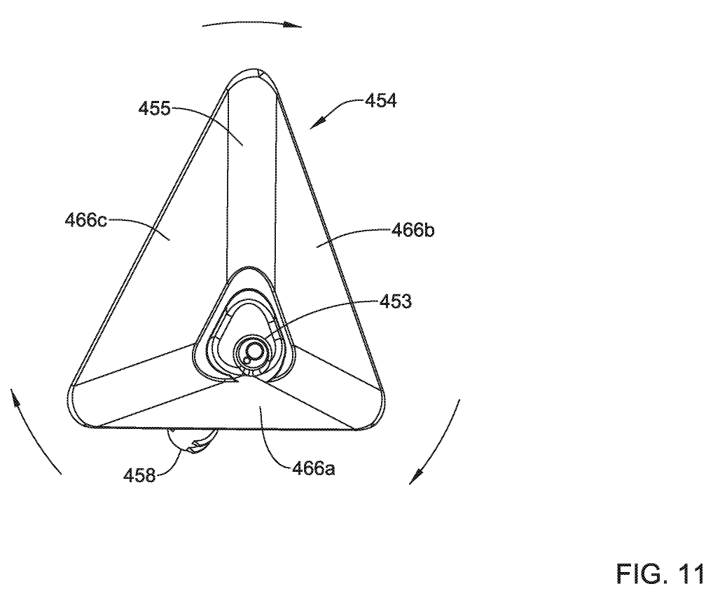

Referring to FIG. 11, the longitudinal axis X of the elongate shaft 453 may be offset from a centroid of the support 454 (e.g., the wedge-shaped body 455) such that the support 454 is eccentrically arranged relative to the longitudinal axis X of the elongate shaft 453. In other words, the centroid of the support 454 may be radially offset or eccentrically positioned relative to the longitudinal axis X of the elongate shaft 453 such that the first plane that the first engagement surface 466a lies in and the second plane that the second engagement surface 466b lies in each intersect the longitudinal axis X at a different longitudinal location, the first plane that the first engagement surface 466a lies in and the third plane that the third engagement surface 466c lies in each intersect the longitudinal axis X at a different longitudinal location, and the second plane that the second engagement surface 466b lies in and the third plane that the third engagement surface 466c lies in each intersect the longitudinal axis X at a different longitudinal location.

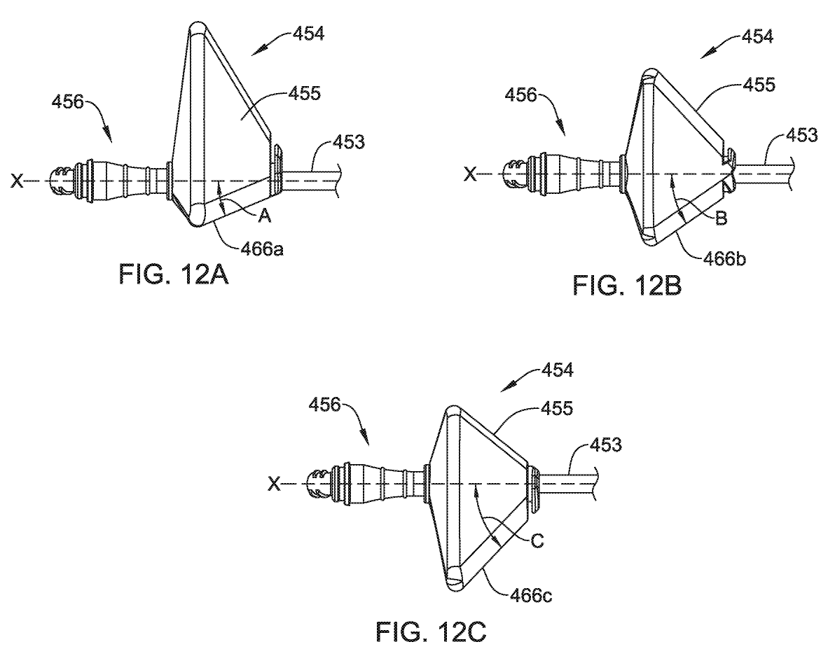

Referring to FIG. 12A, the first engagement surface 466a may extend in a plane that intersects the longitudinal axis X of the elongate shaft 453 at a first fixed acute angle A. In some examples, the angle A between the longitudinal axis X of the vascular access sheath 410 and the skin of the patient that is maintained by the support 454 positioned therebetween may be between about 20 degrees to about 35 degrees, about 20 degrees to about 30 degrees, or about 25 degrees to about 30 degrees, for example. In some instances, the first angle A may be 20 degrees, 25 degrees, 30 degrees, or 35 degrees for example. Furthermore, referring to FIG. 12B, the second engagement surface 466b may extend in a plane that intersects the longitudinal axis X of the elongate shaft 453 at a second fixed acute angle B, different from the first angle A. The second angle B may be greater than the first angle A. In some examples, the angle B between the longitudinal axis X of the vascular access sheath 410 and the skin of the patient that is maintained by the support 454 positioned therebetween may be between about 25 degrees to about 40 degrees, about 25 degrees to about 35 degrees, or about 30 degrees to about 35 degrees, for example. In some instances, the second angle B may be 25 degrees, 30 degrees, 35 degrees, or 40 degrees, for example. Furthermore, referring to FIG. 12C, the third engagement surface 466c may extend in a plane that intersects the longitudinal axis X of the elongate shaft 453 at a third fixed acute angle C, different from the first angle A and the second angle B. The third angle C may be greater than each of the first angle A and the second angle B. In some examples, the angle C between the longitudinal axis X of the vascular access sheath 410 and the skin of the patient that is maintained by the support 454 positioned therebetween may be between about 30 degrees to about 45 degrees, about 35 degrees to about 45 degrees, or about 40 degrees to about 45 degrees, for example. In some instances, the third angle C may be 30 degrees, 35 degrees, 40 degrees, or 45 degrees for example.

During use of the vascular access device 410, the user may determine which of the first, second, and third engagement surfaces 466a/466b/466c to orient towards the skin of the patient to support the hub 456 relative to the skin of the patient in order to provide a desired angle of the hub 456 relative to the skin of the patient. In instances in which the support 454 (e.g., the wedge-shaped body 455) is rotatably fixed relative to the elongate shaft 453, the entire vascular access device 410 may be rotated (either clockwise as shown by the arrows in FIG. 11, or counter-clockwise) about the longitudinal axis X in order to orient the desired one of the first engagement surface 466a, the second engagement surface 466b, or the third engagement surface 466c toward the skin of the patient. In instances in which the support 454 (e.g., the wedge-shaped body 455) is rotatable relative to the elongate shaft 453, such as rotatable around the longitudinal axis X of the elongate shaft 453, the support 454 may be rotated (e.g., clockwise as shown by the arrows in FIG. 11, or in some instances counter-clockwise) to orient the desired one of the first engagement surface 466a, the second engagement surface 466b, or the third engagement surface 466c toward the skin of the patient. Thus, the support 454 may be used to selectively support the hub 456 of the vascular access sheath 410 at any one of a plurality of fixed, stationary angles relative to the skin of the patient.

It is noted that similar to the other embodiments described above, as well as the additional embodiments described below, the vascular access sheath 410 may be provided with one or more openings configured to allow a suture to be passed therethrough for securing the hub 456 to the skin of a patient. The suture openings may be provided in the support 454 or other component of the hub 456.

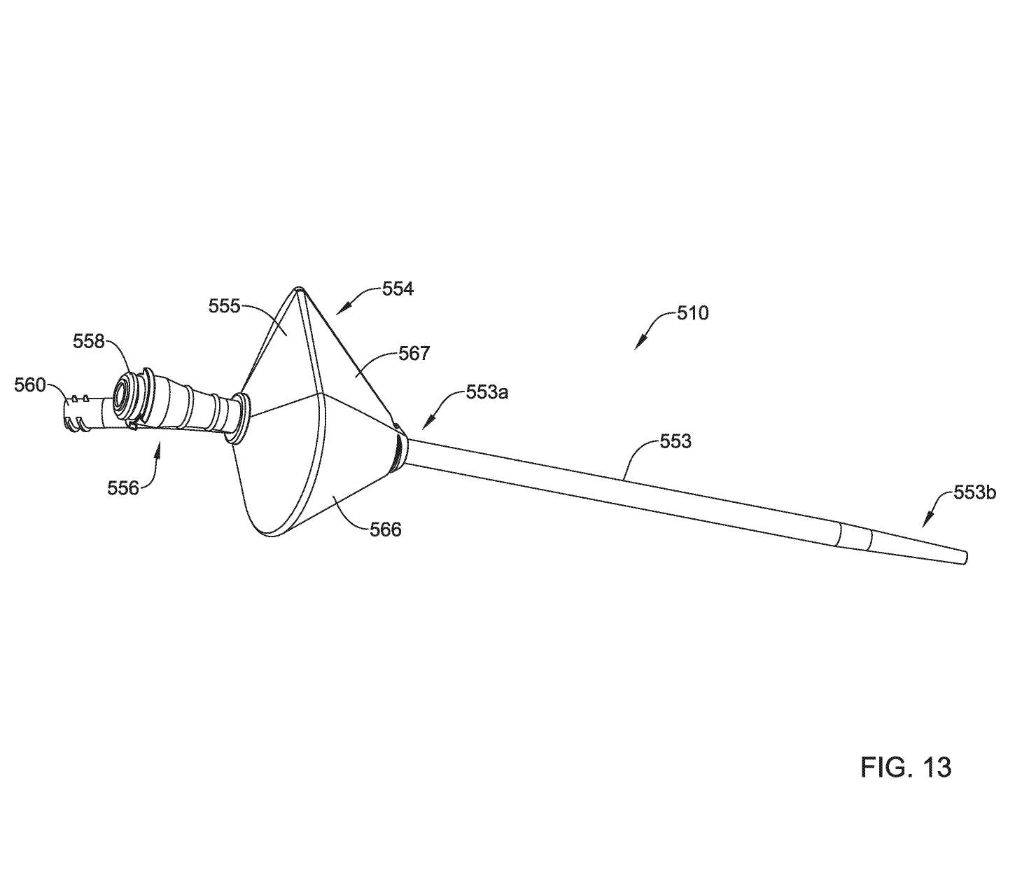

Another embodiment of a vascular access sheath, such as the vascular access sheath 52 described above, incorporating a support for the hub of the vascular access sheath is shown in FIGS. 13, 14 and 15A-15C. A vascular access sheath 510 may include a hub 556 and an elongate shaft 553 extending distally from the hub 556 to a distal end 553b of the elongate shaft 553. A proximal end 553a of the elongate shaft 553 may be fixed to the hub 556 or otherwise secured to the hub 556. The hub 556 may define one or more ports providing fluid communication with one or more lumens extending through the elongate shaft 553. For instance, the hub 556 may include a first port 558 in fluid communication with a first lumen of the elongate shaft 553. In some instances, the hub 556 may include a second port 560, which may be in fluid communication with the first lumen of the elongate shaft 553 or in fluid communication with a second lumen of the elongate shaft 553, if desired.

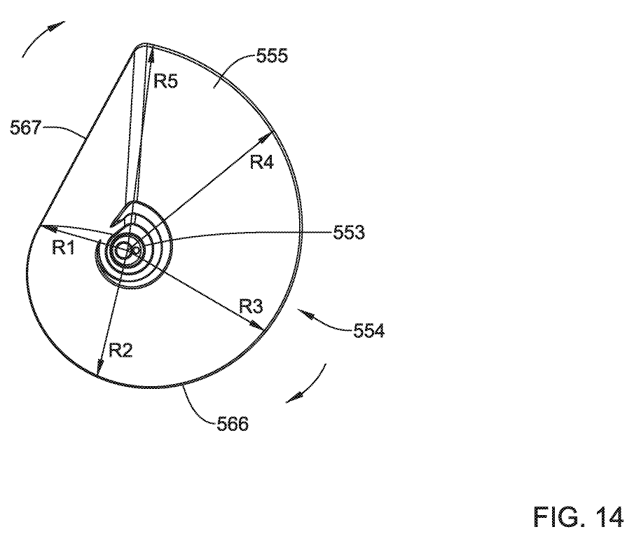

The hub 556 of the vascular access sheath 510 may also include a support 554 configured to rest against (either directly or indirectly) the skin of a patient to maintain the hub 556 at a fixed, stationary acute angle relative to the skin of the patient. In some instances, the support 554 may be fixed relative to the port(s) 558/560 of the hub 556. However, in other instances, the support 554 may be rotatable about the longitudinal axis X of the elongate shaft 553, and thus rotatable relative to the port(s) 558/560. The support 554 may include a wedge-shaped body 555 (e.g., a body tapering from a wider extent to a narrower extent from the longitudinal axis X), which in some instances may be conically shaped body having a spiral base with an outer periphery that extends eccentrically around (e.g., extends spirally around) the longitudinal axis X of the elongate shaft 553. The wedge-shaped body 555 may include a convexly curved engagement surface 566 configured to face and/or engage the skin of the patient to retain the hub 556 at one of a plurality of different acute angles relative to the skin of the patient depending on which region of the curved engagement surface 566 is facing and/or engaged with the skin of the patient. For example, the wedge-shaped body 555 may include or define a curved engagement surface 566 that varies in distance and thus angular orientation relative to the longitudinal axis X in different radial directions from the longitudinal axis X. Turning to FIG. 14, at a given longitudinal location along the longitudinal axis X, the curved engagement surface 566 may extend radially to a plurality of different distances from the longitudinal axis X at different radial directions from the longitudinal axis X. For example, at a first region of the curved engagement surface 566, the radial distance from the longitudinal axis X to the engagement surface 566 may be R1, at a second region of the curved engagement surface 566, the radial distance from the longitudinal axis X to the engagement surface 566 may be R2, at a third region of the curved engagement surface 566, the radial distance from the longitudinal axis X to the engagement surface 566 may be R3, at a fourth region of the curved engagement surface 566, the radial distance from the longitudinal axis X to the engagement surface 566 may be R4, and at a fifth region of the curved engagement surface 566, the radial distance from the longitudinal axis X to the engagement surface 566 may be R5, wherein R2 is greater than R1, R3 is greater than R2, R4 is greater than R3, and R5 is greater than R4. Thus, the support 554 may be configured to face and/or engage the skin of the patient to retain the hub 556 at any one of a plurality of fixed, stationary acute angles relative to the skin of the patient during use of the vascular access device 510 depending on the rotational orientation of the support 554. The wedge-shaped body 555 may be any desired structure that selectively orients the engagement surface 566 at a first acute angle to the longitudinal axis X of the elongate shaft 553 of the vascular access sheath 510, at a second acute angle to the longitudinal axis X of the elongate shaft 553 of the vascular access sheath 510, at a third acute angle to the longitudinal axis X of the elongate shaft 553 of the vascular access sheath 510, or at any one more additional acute angles. Each of the first, second and third acute angles may be different from the others. The first acute angle may be different from each of the second acute angle and the third acute angle, the second acute angle may be different from each of the first acute angle and the third acute angle, and the third acute angle may be different from each of the first acute angle and the second acute angle, providing the vascular access sheath 510 with a degree of variability by permitting a user to select any one of the first acute angle, the second acute angle, the third acute angle, or any one of the addition acute angles. In some instances, the support 554 (e.g., the wedge-shaped body 555) may be rotatably fixed relative to the elongate shaft 553. However, in other instances, the support 554 (e.g., the wedge-shaped body 555) may be rotatable relative to the elongate shaft 553, such as rotatable around the longitudinal axis X of the elongate shaft 553.

Referring to FIG. 14, the longitudinal axis X of the elongate shaft 453 may be offset from a centroid of the support 554 (e.g., the wedge-shaped body 555) such that the support 554 is eccentrically arranged relative to the longitudinal axis X of the elongate shaft 553. In other words, the centroid of the support 554 may be radially offset or eccentrically positioned relative to the longitudinal axis X of the elongate shaft 553. In some instances, the support 554 may include a planar surface 567 extending from the greatest outer extent of the curved engagement surface 566 to the least outer extent of the curved engagement surface 566.

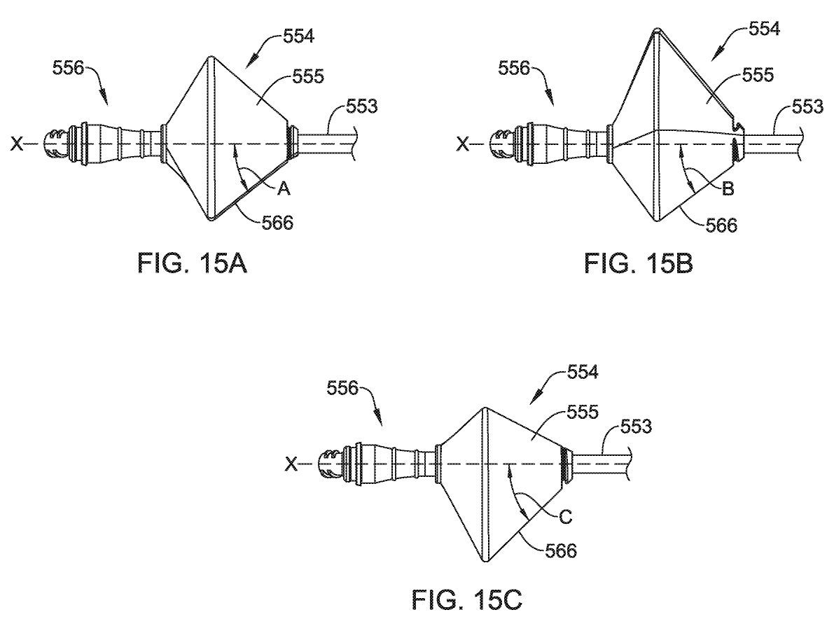

Referring to FIG. 15A, a first region of the engagement surface 566 located at a first radial direction may extend tangent to a plane that intersects the longitudinal axis X of the elongate shaft 553 at a first fixed acute angle A. In some examples, the angle A between the longitudinal axis X of the vascular access sheath 510 and the skin of the patient that is maintained by the support 554 positioned therebetween may be between about 20 degrees to about 35 degrees, about 20 degrees to about 30 degrees, or about 25 degrees to about 30 degrees, for example. In some instances, the first angle A may be 20 degrees, 25 degrees, 30 degrees, or 35 degrees for example. Furthermore, referring to FIG. 15B, a second region of the engagement surface 566 located at a second radial direction may extend tangent to a plane that intersects the longitudinal axis X of the elongate shaft 553 at a second fixed acute angle B, different from the first angle A. The second angle B may be greater than the first angle A. In some examples, the angle B between the longitudinal axis X of the vascular access sheath 510 and the skin of the patient that is maintained by the support 554 positioned therebetween may be between about 25 degrees to about 40 degrees, about 25 degrees to about 35 degrees, or about 30 degrees to about 35 degrees, for example. In some instances, the second angle B may be 25 degrees, 30 degrees, 35 degrees, or 40 degrees, for example. Furthermore, referring to FIG. 15C, a third region of the engagement surface 566 may extend tangent to a plane that intersects the longitudinal axis X of the elongate shaft 553 at a third fixed acute angle C, different from the first angle A and the second angle B. The third angle C may be greater than each of the first angle A and the second angle B. In some examples, the angle C between the longitudinal axis X of the vascular access sheath 510 and the skin of the patient that is maintained by the support 554 positioned therebetween may be between about 30 degrees to about 45 degrees, about 35 degrees to about 45 degrees, or about 40 degrees to about 45 degrees, for example. In some instances, the third angle C may be 30 degrees, 35 degrees, 40 degrees, or 45 degrees for example.

It is noted, with the curved engagement surface 566 (which may have a continuously changing radii from the longitudinal axis X around the perimeter of the support 554), the angle between the longitudinal axis X of the vascular access sheath 510 and the skin of the patient may be any angle between the smallest angle permitted by the support 554 and the greatest angle permitted by the support 554 depending on the rotational orientation of the support 554 and/or the hub 556 of the vascular access sheath 510.

During use of the vascular access device 510, the user may determine which region of the curved engagement surface 566 to orient towards the skin of the patient to support the hub 556 relative to the skin of the patient in order to provide a desired angle of the hub 556 relative to the skin of the patient. In instances in which the support 554 (e.g., the wedge-shaped body 555) is rotatably fixed relative to the elongate shaft 553, the entire vascular access device 510 may be rotated about the longitudinal axis X in order to orient the desired region of the curved engagement surface 566 toward the skin of the patient. In instances in which the support 554 (e.g., the wedge-shaped body 555) is rotatable relative to the elongate shaft 553, such as rotatable around the longitudinal axis X of the elongate shaft 553, the support 554 may be rotated to orient the desired region of the curved engagement surface 566 toward the skin of the patient. Thus, the support 554 may be used to selectively support the hub 556 of the vascular access sheath 510 at any one of a plurality of fixed, stationary angles relative to the skin of the patient.

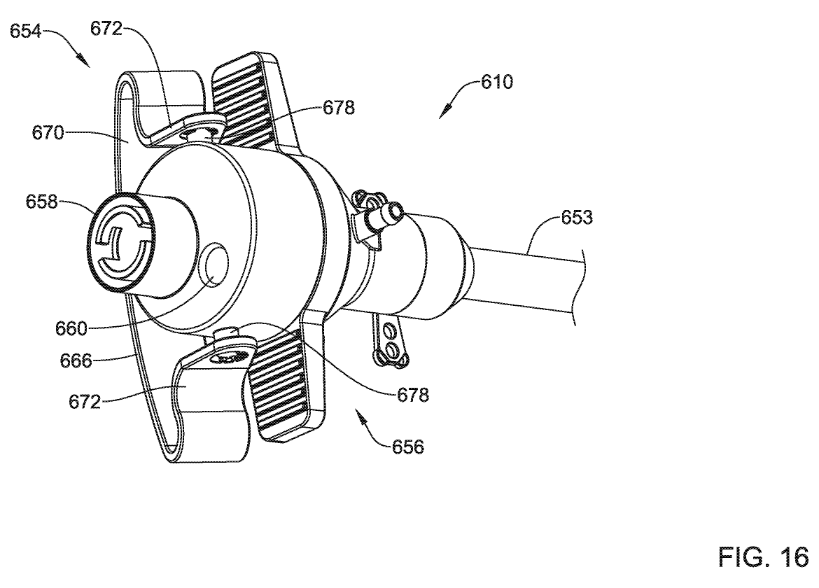

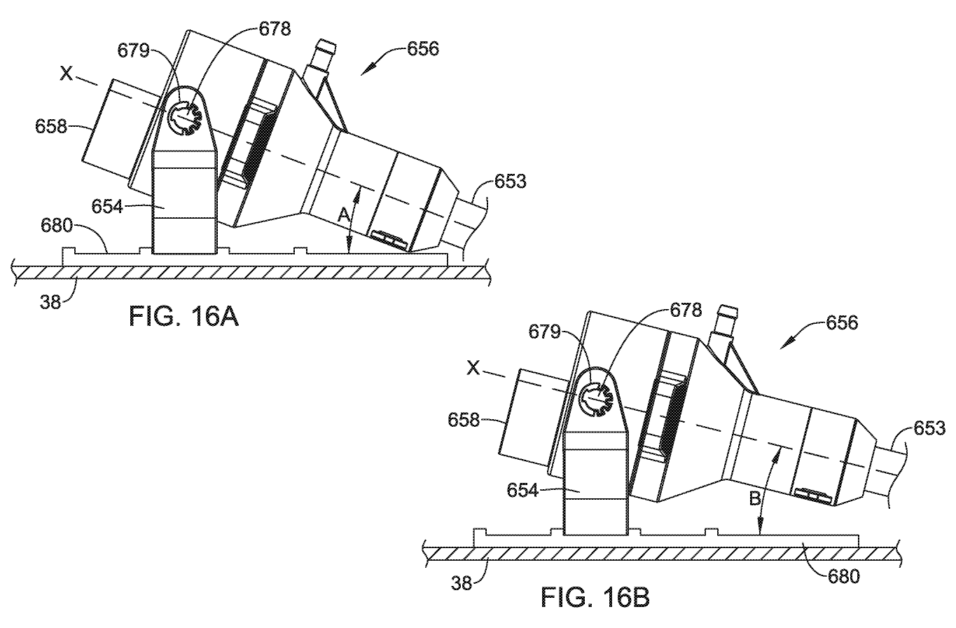

Another embodiment of a vascular access sheath, such as the vascular access sheath 52 described above, incorporating a support for the hub of the vascular access sheath is shown in FIGS. 16, 16A and 16B. A vascular access sheath 610 may include a hub 656 and an elongate shaft 653 extending distally from the hub 656 to a distal end (not shown) of the elongate shaft 653. A proximal end of the elongate shaft 653 may be fixed to the hub 656 or otherwise secured to the hub 656. The hub 656 may define one or more ports providing fluid communication with one or more lumens extending through the elongate shaft 653. For instance, the hub 656 may include a first port 658 in fluid communication with a first lumen of the elongate shaft 653. In some instances, the hub 656 may include a second port 660, which may be in fluid communication with the first lumen of the elongate shaft 653 or in fluid communication with a second lumen of the elongate shaft 653, if desired.

The hub 656 of the vascular access sheath 610 may also include a support 654 configured to rest against (either directly or indirectly) the skin of a patient to maintain the hub 656 at a fixed, stationary acute angle relative to the skin of the patient. In some instances, the support 654 may be fixed relative to the port(s) 658/660 of the hub 656. However, in other instances, the support 654 may be pivotable relative to the port(s) 658/660. The support 654 may include a base 670 including an engagement surface 666 configured to face and/or engage the skin of the patient to retain the hub 656 at a first fixed, stationary acute angle relative to the skin of the patient during use of the vascular access device 610. The support 654 may also include a first arm and/or a second arm 672 extending from the base 670. For example, the support 654 may include first and second arms 672 positioned on opposing sides of the body of the hub 656 (e.g., on opposing sides of the ports(s) 658/660). The arms 672 may be pivotably coupled to the body of the hub 656, such as via first and second pins 678 extending in opposite directions from the body of the hub 656 along a pivot axis. In some instances, the pivot axis may be perpendicular to the longitudinal axis X of the elongate shaft 653. The pins 678 may be rotatably engaged within apertures 679 of the arms 672 of the support 654. The support 654 may be pivotable around the pins 678 to position the support 654 at any one of a plurality of angular positions relative to the longitudinal axis X of the elongate shaft 653. In some instances, the support 654 may be pivotable to an infinite quantity of angular positions, while in other embodiments the support 654 may be pivotable between a plurality of discrete, incremental positions. For example, in some instances, the pins 678 and/or the apertures 679 may include one or more, or a plurality of notches, grooves, cogs, teeth, etc. configured to retain the support 654 at one of a plurality of incremental angular positions relative to the longitudinal axis X. In some instances, the arms 672 may be deflected inward toward the hub 656, such as in a direction generally transverse to the longitudinal axis X, to disengage the cogs or teeth (or other engagement features) on the pins 678 from the mating notches (or other engagement features) of the apertures 679, to allow pivoting of the support 654 around the pins 678. Once a desired angular position of the support 654 is achieved, the arms 672 may be released to allow the arms 672 to move away from the hub 656 in order to revert back to their engaged position in which the cogs or teeth (or other engagement features) of the pins 678 are engaged with the notches (or other engagement features) of the apertures 679 to retain the support 654 at the desired angular position.

FIG. 16A illustrates the support 654 in a first angular configuration and/or position, and FIG. 16B illustrates the support 654 in a second angular configuration and/or position. Furthermore, FIGS. 16A and 16B illustrate the support 654 may include or be used with a base plate 680, which in some instances may be an adhesive patch or strip, adhered to the patient’s skin 38 proximate the access site. The base plate 680 may provide a retention surface that the engagement surface 666 of the support 654 contacts when the hub 656 is being held stationary during use. In some instances, the retention surface may include one or more, or a plurality of ribs, cleats, or other features that the base 670 of the support 654 can be propped or otherwise engaged against. In some instances, the base 670 of the support 654 may provide sufficient contact area to directly contact and engage the skin of the patient. Thus, in some instances, the base plate 680 or other patch, strip, etc. may not be required.

FIG. 16A illustrates the engagement surface 666 facing and/or engaged with the skin of the patient to retain the hub 656 at a first fixed, stationary acute angle A relative to the skin 38 of the patient during use of the vascular access device 610 in a first configuration and/or position. FIG. 16B illustrates the engagement surface 666 facing and/or engaged with the skin 38 of the patient to retain the hub 656 at a second fixed, stationary acute angle B relative to the skin 38 of the patient during use of the vascular access device 610 in a second configuration and/or position. The selected pivotable position of the support 654 may determine the angle of the hub 656 relative to the skin 38 of the patient. The first acute angle A may be different from the second acute angle B, providing the vascular access sheath 610 with a degree of variability by permitting a user to select the desired acute angle.

In some examples, the angle A between the longitudinal axis X of the vascular access sheath 610 and the skin 38 of the patient that is maintained by the support 654 positioned therebetween may be between about 20 degrees to about 45 degrees, about 20 degrees to about 40 degrees, or about 30 degrees to about 40 degrees, for example. In some instances, the first angle A may be 30 degrees, 35 degrees or 40 degrees, for example. In some examples, the angle B between the longitudinal axis X of the vascular access sheath 610 and the skin 38 of the patient that is maintained by the support 654 positioned therebetween may be between about 20 degrees to about 45 degrees, about 30 degrees to about 45 degrees, or about 35 degrees to about 45 degrees, for example. In some instances, the second angle B may be 35 degrees, 40 degrees, or 45 degrees, for example. During use of the vascular access device 610, the user may determine which pivotable position to position the support 654 at in order to provide a desired angle of the hub 656 relative to the skin of the patient. Thus, the support 654 may be used to selectively support the hub 656 of the vascular access sheath 610 at any one of a plurality of fixed, stationary angles relative to the skin of the patient.

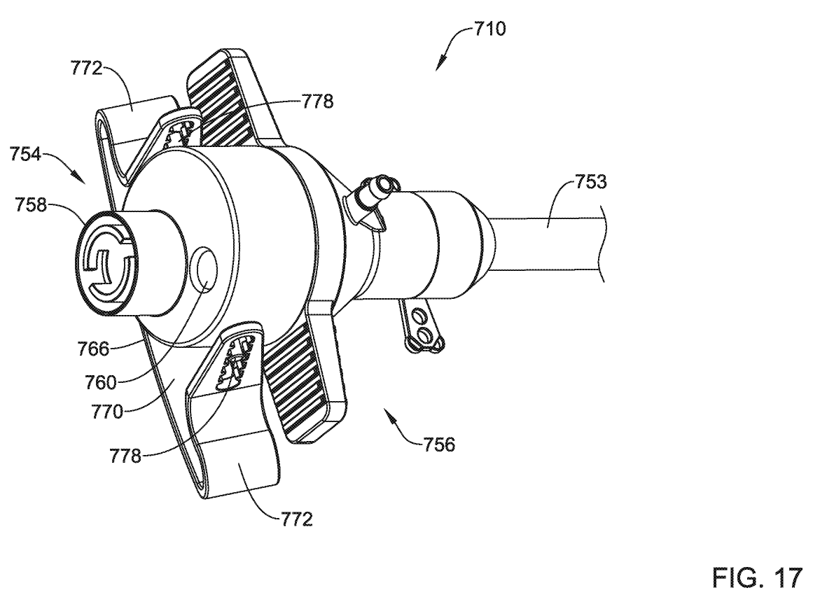

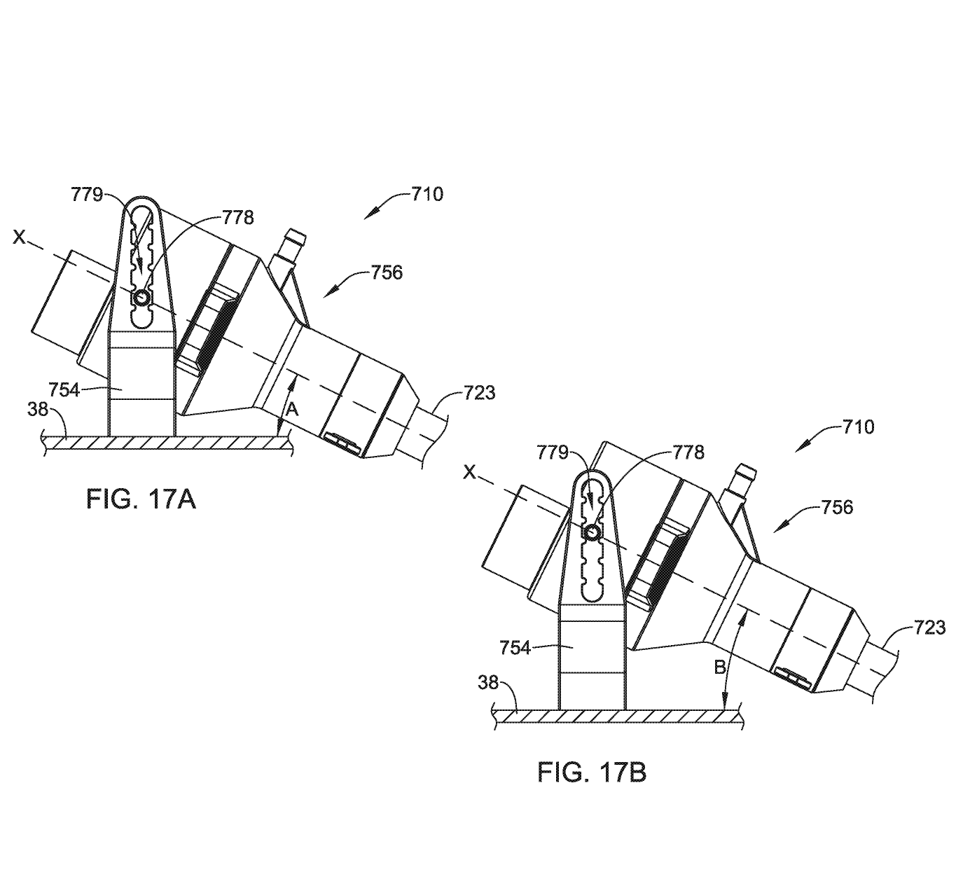

Another embodiment of a vascular access sheath, such as the vascular access sheath 52 described above, incorporating a support for the hub of the vascular access sheath is shown in FIGS. 17, 17A and 17B. A vascular access sheath 710 may include a hub 756 and an elongate shaft 753 extending distally from the hub 756 to a distal end (not shown) of the elongate shaft 753. A proximal end of the elongate shaft 753 may be fixed to the hub 756 or otherwise secured to the hub 756. The hub 756 may define one or more ports providing fluid communication with one or more lumens extending through the elongate shaft 753. For instance, the hub 756 may include a first port 758 in fluid communication with a first lumen of the elongate shaft 753. In some instances, the hub 756 may include a second port 760, which may be in fluid communication with the first lumen of the elongate shaft 753 or in fluid communication with a second lumen of the elongate shaft 753, if desired.

The hub 756 of the vascular access sheath 710 may also include a support 754 configured to rest against (either directly or indirectly) the skin of a patient to maintain the hub 756 at a fixed, stationary acute angle relative to the skin of the patient. In some instances, the support 754 may be fixed relative to the port(s) 758/760 of the hub 756. However, in other instances, the support 754 may be pivotable and/or translatable relative to the port(s) 758/760. The support 754 may include a base 770 including an engagement surface 766 configured to face and/or engage the skin of the patient to retain the hub 756 at a first fixed, stationary acute angle relative to the skin of the patient during use of the vascular access device 710. The support 754 may also include a first arm and/or a second arm 772 extending from the base 770. For example, the support 754 may include first and second arms 772 positioned on opposing sides of the body of the hub 756 (e.g., on opposing sides of the ports(s) 758/760). The arms 772 may be pivotably coupled to the body of the hub 756 and/or translatably (e.g., slidably) coupled to the body of the hub 756, such as via first and second pins 778 extending in opposite directions from the body of the hub 756 along an axis (e.g., a pivot axis). In some instances, the pivot axis may be perpendicular to the longitudinal axis X of the elongate shaft 753. The pins 778 may be rotatably and/or slidably engaged within elongated apertures 779 (e.g., elongated slots) of the arms 772 of the support 754. The support 754 may be pivotable around the pins 778 and/or translatable along the elongated apertures 779 (e.g., elongated slots) to position the support 754 at any one of a plurality of positions. In some instances, the arms 772 of the support 754 may be translatable relative to the body of the hub 756 via moving the pins 778 in the elongated slots 779 to an infinite quantity of positions, while in other embodiments the arms 772 of the support 754 may be translatable relative to the body of the hub 756 via moving the pins 778 in the elongated slots 779 between a plurality of discrete, incremental positions. For example, in some instances, the pins 778 and/or the elongated apertures 779 (e.g., elongated slots) may include one or more, or a plurality of notches, grooves, cogs, teeth, etc. configured to retain the support 754 at one of a plurality of incremental positions relative to the body of the hub 756.

FIG. 17A illustrates the support 754 in a first configuration and/or position, and FIG. 17B illustrates the support 754 in a second configuration and/or position. Furthermore, in some instances the support 754 may include or be used with a base plate, similar to the base plate 680 of FIGS. 16A and 16B, which in some instances may be an adhesive patch or strip, adhered to the patient’s skin 38 proximate the access site. The base plate may provide a retention surface that the engagement surface 766 of the support 754 contacts when the hub 756 is being held stationary during use. In some instances, the retention surface may include one or more, or a plurality of ribs or other features that the base 770 of the support 754 can be propped or engaged against.

FIG. 17A illustrates the engagement surface 766 facing and/or engaged with the skin 38 of the patient to retain the hub 756 at a first fixed, stationary acute angle A relative to the skin 38 of the patient during use of the vascular access device 710 in a first configuration and/or position. FIG. 17B illustrates the engagement surface 766 facing and/or engaged with the skin of the patient to retain the hub 756 at a second fixed, stationary acute angle B relative to the skin 38 of the patient during use of the vascular access device 710 in a second configuration and/or position. The selected position of the support 754 may determine the angle of the hub 756 relative to the skin 38 of the patient. The first acute angle A may be different from the second acute angle B, providing the vascular access sheath 710 with a degree of variability by permitting a user to select the desired acute angle.

In some examples, the angle A between the longitudinal axis X of the vascular access sheath 710 and the skin 38 of the patient that is maintained by the support 754 positioned therebetween may be between about 20 degrees to about 45 degrees, about 20 degrees to about 40 degrees, or about 30 degrees to about 40 degrees, for example. In some instances, the first angle A may be 30 degrees, 35 degrees or 40 degrees, for example. In some examples, the angle B between the longitudinal axis X of the vascular access sheath 710 and the skin 38 of the patient that is maintained by the support 754 positioned therebetween may be between about 20 degrees to about 45 degrees, about 30 degrees to about 45 degrees, or about 35 degrees to about 45 degrees, for example. In some instances, the second angle B may be 35 degrees, 40 degrees, or 45 degrees, for example. During use of the vascular access device 710, the user may determine which position to position the support 754 at in order to provide a desired angle of the hub 756 relative to the skin of the patient. Thus, the support 754 may be used to selectively support the hub 756 of the vascular access sheath 710 at any one of a plurality of fixed, stationary angles relative to the skin of the patient.

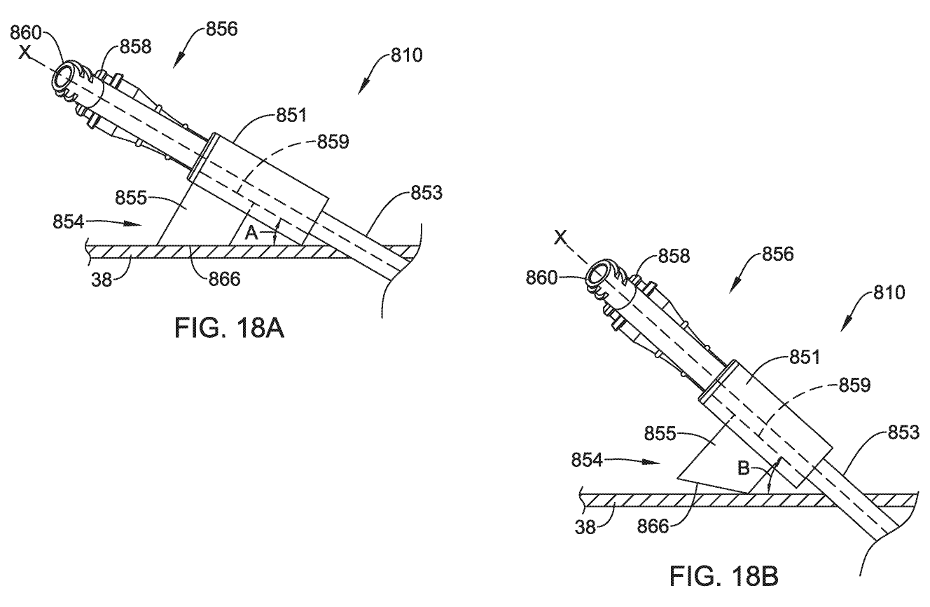

Another embodiment of a vascular access sheath, such as the vascular access sheath 52 described above, incorporating a support for the hub of the vascular access sheath is shown in FIGS. 18A and 18B. A vascular access sheath 810 may include a hub 856 and an elongate shaft 853 extending distally from the hub 856 to a distal end (not shown) of the elongate shaft 853. A proximal end of the elongate shaft 853 may be fixed to the hub 856 or otherwise secured to the hub 856. The hub 856 may define one or more ports providing fluid communication with one or more lumens extending through the elongate shaft 853. For instance, the hub 856 may be similar to one of the other hubs disclosed herein, including a first port in fluid communication with a first lumen of the elongate shaft 853, and in some instances may also include a second port in fluid communication with the first lumen of the elongate shaft 853 or in fluid communication with a second lumen of the elongate shaft 853, if desired.

The hub 856 of the vascular access sheath 810 may also include a support 854 configured to rest against (either directly or indirectly) the skin 38 of a patient to maintain the hub 856 at a fixed, stationary acute angle relative to the skin of the patient. In some instances, the support 854 may be fixed relative to the port(s) 858/860 of the hub 856. However, in other instances, the support 854 may be slidable relative to the port(s) 858/860. For example, the support 854 may be slidable along a channel 859 formed in the body of the hub 856 between any one of a plurality of positions. In some instances, the support 854 may be slidable in a direction generally or substantially parallel to the longitudinal axis X of the elongate shaft 853. The support 854 may include a wedge-shaped body 855 (e.g., a body tapering from a wider extent to a narrower extent from the longitudinal axis X) and an engagement surface 866 configured to face and/or engage the skin of the patient to retain the hub 856 at a first fixed, stationary acute angle relative to the skin of the patient during use of the vascular access device 810. The support 854 may be slidable along the channel 859 to position the support 854 at any one of a plurality of positions relative to the body of the hub 856. In some instances, the support 854 may be slidable to an infinite quantity of positions, while in other embodiments the support 854 may be slidable between a plurality of discrete, incremental positions. For example, in some instances, channel 859 may include one or more, or a plurality of notches, grooves, cogs, teeth, etc. configured to retain the support 854 at one of a plurality of incremental positions relative to the body of the hub 856.

FIG. 18A illustrates the support 854 in a first configuration and/or position, and FIG. 18B illustrates the support 854 in a second configuration and/or position. FIG. 18A illustrates the engagement surface of the support 854 facing and/or engaged with the skin 38 of the patient to retain the hub 856 at a first fixed, stationary acute angle A relative to the skin 38 of the patient during use of the vascular access device 810 in a first configuration and/or position. FIG. 18B illustrates the engagement surface 866 of the support 854 facing and/or engaged with the skin 38 of the patient to retain the hub 856 at a second fixed, stationary acute angle B relative to the skin 38 of the patient during use of the vascular access device 810 in a second configuration and/or position. The selected position of the support 854 in the channel 859 may determine the angle of the hub 856 relative to the skin 38 of the patient. The first acute angle A may be different from the second acute angle B, providing the vascular access sheath 810 with a degree of variability by permitting a user to select the desired acute angle.

In some examples, the angle A between the longitudinal axis X of the vascular access sheath 810 and the skin of the patient that is maintained by the support 854 positioned therebetween may be between about 20 degrees to about 45 degrees, about 20 degrees to about 40 degrees, or about 30 degrees to about 40 degrees, for example. In some instances, the first angle A may be 30 degrees, 35 degrees or 40 degrees, for example. In some examples, the angle B between the longitudinal axis X of the vascular access sheath 810 and the skin of the patient that is maintained by the support 854 positioned therebetween may be between about 20 degrees to about 45 degrees, about 30 degrees to about 45 degrees, or about 35 degrees to about 45 degrees, for example. In some instances, the second angle B may be 35 degrees, 40 degrees, or 45 degrees, for example. During use of the vascular access device 810, the user may determine which position to position the support 854 within the channel 859 in order to provide a desired angle of the hub 856 relative to the skin 38 of the patient. Thus, the support 854 may be used to selectively support the hub 856 of the vascular access sheath 810 at any one of a plurality of fixed, stationary angles relative to the skin 38 of the patient.

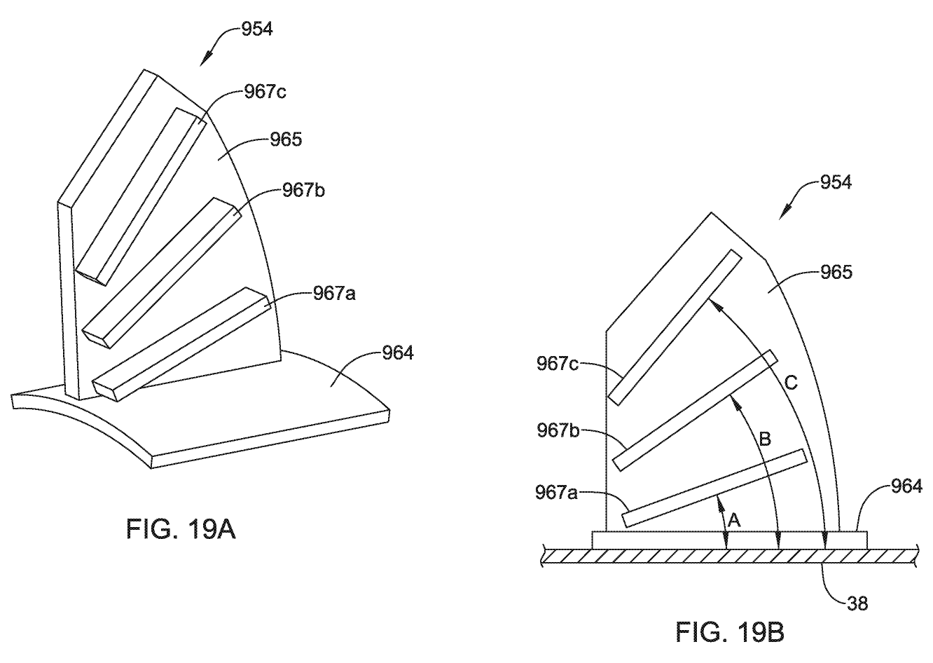

FIGS. 19A and 19B illustrate another support 954 which may be configured to rest against (either directly or indirectly) the skin of a patient to maintain the hub of a vascular access sheath (such as the vascular access sheath 52 or another vascular access sheath described herein) at a fixed, stationary acute angle relative to the skin of the patient. The support 954 may include a base 964 having an engagement surface configured to face and/or engage the skin of the patient and a beam 965 extending upward from the base 964. The beam 965 may include one or more, or a plurality of retention features defining incremental positions for retaining the hub of a vascular access sheath at a fixed, stationary acute angle relative to the skin of the patient during use. For example, the support 954 may include a plurality of cleats 967a, 967b, 967c extending from the beam 965 forming ledges for holding the hub of the vascular access sheath. In other instances, the hub of the vascular access sheath may be coupled to, propped against, or otherwise engaged with one of the cleats 967a, 967b, 967c.

The first cleat 967a may extend at a first fixed acute angle A relative to the skin 38 of the patient. In instances in which the hub of the vascular access sheath is supported by the first cleat 967a, the hub of the vascular access sheath may be held stationary at the angle A between the longitudinal axis of the vascular access sheath and the skin of the patient. In some instances, the angle A may be between about 20 degrees to about 35 degrees, about 20 degrees to about 30 degrees, or about 25 degrees to about 30 degrees, for example. In some instances, the first angle A may be 20 degrees, 25 degrees, 30 degrees, or 35 degrees for example. The second cleat 967b may extend at a second fixed acute angle B relative to the skin 38 of the patient. In instances in which the hub of the vascular access sheath is supported by the second cleat 967b, the hub of the vascular access sheath may be held stationary at the angle B between the longitudinal axis of the vascular access sheath and the skin of the patient. The second fixed acute angle B may be different from the first angle A. For example, the second angle B may be greater than the first angle A. In some instances, the angle B may be between about 25 degrees to about 40 degrees, about 25 degrees to about 35 degrees, or about 30 degrees to about 35 degrees, for example. In some instances, the second angle B may be 25 degrees, 30 degrees, 35 degrees, or 40 degrees, for example. The third cleat 967c may extend at a third fixed acute angle C relative to the skin 38 of the patient. In instances in which the hub of the vascular access sheath is supported by the third cleat 967c, the hub of the vascular access sheath may be held stationary at the angle C between the longitudinal axis of the vascular access sheath and the skin of the patient. The third angle C may be different from the first angle A and the second angle B. For example, the third angle C may be greater than each of the first angle A and the second angle B. In some examples, the angle C may be between about 30 degrees to about 45 degrees, about 35 degrees to about 45 degrees, or about 40 degrees to about 45 degrees, for example. In some instances, the third angle C may be 30 degrees, 35 degrees, 40 degrees, or 45 degrees for example.

During use of the vascular access device, the user may determine which of the first, second, and third cleats 967a, 967b, 967c to retain the hub of the vascular access device relative thereto in order to provide a desired angle of the hub relative to the skin of the patient. Thus, the support 954 may be used to selectively support the hub of the vascular access sheath at any one of a plurality of fixed, stationary angles relative to the skin of the patient.

It is noted that the various supports disclosed herein may be provided preassembled with the hub of the vascular access sheath, may be provided in a kit with the vascular access sheath, and/or may be provided as an accessory to the vascular access sheath, as desired. Thus, the supports may be used as needed during use of the vascular access sheath.

The materials that can be used for the various components of the vascular access sheaths, and relative components may include those commonly associated with medical devices. For example, some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), high-performance sysethylene vinyl acetate copolymers (EVA), silicones, polyethylene (PE), FFKM or perfluoroelastomer which is a high-performance synthetic rubber, ethylene propylene diene monomer (EPDM), thermoplastic vulcanizates (TPV), thermoplastic elastomer (TPE), a copolymer of tetrafluoroethylene and propylene (TFEP) also known as Aflas, high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in detail, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A vascular access sheath, comprising:
an elongate tubular shaft having a proximal end, a distal end, and a lumen extending therethrough along a longitudinal axis of the elongate shaft;
a hub secured to the proximal end of the elongate tubular shaft, the hub including an access port in communication with the lumen of the elongate shaft, and
a support including an engagement surface configured to rest directly or indirectly against the skin of a patient to maintain the hub at a fixed, stationary acute angle relative to the skin of the patient.

2. The vascular access sheath of claim 1, wherein the support is rotatable about the longitudinal axis relative to the port of the hub.

3. The vascular access sheath of claim 1, wherein the support is fixed relative to the port of the hub.

4. The vascular access sheath of claim 1, wherein the support includes a wedge-shaped body tapering from a wider extent to a narrower extent from the longitudinal axis.

5. The vascular access sheath of claim 4, wherein the engagement surface lies in a plane extending at an acute angle to the longitudinal axis.

6. The vascular access sheath of claim 4, wherein the support includes a first wing extending from the wedge-shaped body in a first direction and a second wing extending from the wedge-shaped body in a second, different direction.

7. The vascular access sheath of claim 4, wherein the engagement surface is a first engagement surface extending at a first acute angle relative to the longitudinal axis, wherein the support includes a second engagement surface extending at a second acute angle relative to the longitudinal axis, the second angle being different from the first acute angle.

8. The vascular access sheath of claim 7, wherein the support includes a third engagement surface extending at a third acute angle relative to the longitudinal axis, the third angle being different from the first and second acute angles.

9. The vascular access sheath of claim 8, wherein the support is pyramidal shaped having a triangular base, wherein the first engagement surface extends from a first side of the triangular base, the second engagement surface extends from a second side of the triangular base, and the third engagement surface extends from a third side of the triangular base.

10. The vascular access sheath of claim 1, wherein the support includes a conically shaped body having a spiral base with an outer periphery that extends eccentrically around the longitudinal axis of the elongate shaft.

11. The vascular access sheath of claim 10, wherein the engagement surface is a convexly curved engagement surface that varies in distance relative to the longitudinal axis in different radial directions from the longitudinal axis of the elongate shaft.

12. The vascular access sheath of claim 1, wherein the support is pivotable relative to the port of the hub about a pivot axis extending perpendicular to the longitudinal axis.

13. The vascular access sheath of claim 1, wherein the support includes first and second arms positioned on opposite sides of the hub.

14. The vascular access sheath of claim 13, wherein the first and second arms each include an elongate slot, and the hub includes first and second pins extending in opposite directions from the hub, the first pin extending into the elongate slot of the first arm and the second pin extending into the elongate slot of the second arm.

15. The vascular access sheath of claim 1, wherein the support is slidably disposed within a channel of the hub and movable in a direction generally parallel to the longitudinal axis.

16. A vascular access sheath, comprising:
an elongate tubular shaft having a proximal end, a distal end, and a lumen extending therethrough along a longitudinal axis of the elongate shaft;
a hub secured to the proximal end of the elongate tubular shaft, the hub including an access port in communication with the lumen of the elongate shaft, and
a support provided with the hub, the support including an engagement surface extending at an acute angle to the longitudinal axis of the elongate shaft, the engagement surface configured to rest directly or indirectly against the skin of a patient to maintain the hub at a fixed, stationary acute angle relative to the skin of the patient.

17. The vascular access sheath of claim 16, wherein the support includes a wedge-shaped body tapering from a wider extent from the longitudinal axis proximate a proximal end of the wedge-shaped body to a narrower extent from the longitudinal axis proximate a distal end of the wedge-shaped body.

18. The vascular access sheath of claim 16, wherein the support is rotatable about the longitudinal axis relative to the port of the hub.

19. A method of using a vascular access sheath, the method comprising:
advancing an elongate tubular shaft of the vascular access sheath into a blood vessel of a patient, the vascular access sheath including a hub secured to a proximal end of the elongate tubular shaft and a support provided with the hub; and
positioning an engagement surface of the support directly or indirectly against the skin of the patient to maintain the hub at a fixed, stationary acute angle relative to the skin of the patient.

20. The method of claim 19, wherein the support includes a wedge-shaped body.
